(19) United States
(12) Patent Application Publication
(10) Pub. No.: US 2016/0089443 A1
(43) Pub. Date: Mar. 31, 2016

(54) PROTEIN-GRAPHENE NANOCOMPOSITE DRUG CARRIER

(71) Applicant: NATIONAL CHIAO TUNG UNIVERSITY, Hsinchu City (TW)

(72) Inventors: Shang-Hsiu HU, Taipei City (TW); San-Yuan CHEN, Hsinchu City (TW); Yen-Ho LAI, Chiayi County (TW); Chih-Sheng CHIANG, Taichung City (TW); Min-Yu CHIANG, Taitung County (TW)

(21) Appl. No.: 14/614,391

(22) Filed: Feb. 4, 2015

(30) Foreign Application Priority Data
Sep. 30, 2014 (TW) 103134041

(51) Int. Cl. A61K 47/42; A61K 47/06; A61K 31/121; A61K 31/337; A61K 31/704; A61K 33/24

(52) U.S. Cl. CPC A61K 47/42; A61K 31/704; A61K 33/24; A61K 31/121; A61K 31/337; A61K 47/06

(57) ABSTRACT

A drug carrier includes an aqueous solution, a protein shell, graphenes, and a bioactive agent. The protein shell encloses the aqueous solution, and includes at least one hydrophilic/hydrophobic layer. The graphenes are dispersed in the protein shell, and the bioactive agent is in the aqueous solution and/or the protein shell.

### RELATED APPLICATIONS

This application claims priority to Taiwanese Application Serial Number 103134041, filed Sep. 30, 2014, which is herein incorporated by reference.

### BACKGROUND

1. Field of Invention

The present invention relates to a drug carrier. More particularly, the present invention relates to a drug carrier with the shell including at least protein and graphene.

2. Description of Related Art

Most of common drug carriers are composed of inorganic or polymer materials, which have to be modified or grafted with other molecules to reduce the phagocytosis of the drug carrier by immune cells or proteins, and to increase the probability of the drug carrier being delivered to the designated therapeutic areas. Although the inorganic or polymer materials can flexibly manipulate the characteristic as the drug carrier by modification, they also have disadvantage of being susceptible to temperature and the surrounding pH value. Further, most of the inorganic or polymer carriers have insufficient biological compatibility, which limits the development of such carriers.

In addition, most of the drug carriers have poor structural stability, so drugs encapsulated in the drug carriers may have been released during the delivery before the drug carriers reached the designated therapeutic areas. As such, the drugs cannot be accurately delivered to the designated therapeutic areas, and it is difficult to know the administrated dosage and results in poor therapeutic effects. In case of effective controlling of timing and amount of drug release, the aforementioned problems may be solved and the therapeutic effects can be improved. However, typical drug carriers do not have such drug releasing mechanism.

Accordingly, there is a need for a drug carrier in the drug administration of disease treatments that the drug carrier can avoid attack and phagocytosis of immune cells or proteins, and has specificity, high stability, and controllable timing and amount of drug release to improve the therapeutic effects.

### SUMMARY

The present invention combines protein and graphene in a single carrier, which is a protein nanocomposite drug carrier with dual therapy of both chemotherapy drug delivery and photothermal therapy. The carrier with protein and graphene may combine with iron oxide particle, and a magnetic protein nanocomposite drug carrier with dual-targeted therapy of both chemical and physical.

An aspect of the present invention provides a drug carrier, including an aqueous solution, a protein shell, graphenes, and a bioactive agent. The protein shell encloses the aqueous solution, and includes at least one hydrophilic/hydrophobic layer. The graphenes are dispersed in the protein shell, and the bioactive agent is in the aqueous solution and/or the protein shell.

According to one embodiment of the present invention, the protein shell is made of a protein, which is amphiphilic lactoferrin, albumin or silk protein.

According to one embodiment of the present invention, the protein has a concentration of about 1-5 wt % in the drug carrier.

According to one embodiment of the present invention, the graphenes have a concentration of about 0.01-4 wt % in the drug carrier.

According to one embodiment of the present invention, the graphenes are reduced graphene oxides.

According to one embodiment of the present invention, the graphenes have a diameter of about 20-400 nm.

According to one embodiment of the present invention, the drug carrier further includes iron oxide dispersed in the protein shell.

According to one embodiment of the present invention, the drug carrier has a diameter of about 100-4000 nm.

According to one embodiment of the present invention, the bioactive agent in the aqueous solution is a hydrophilic agent.

According to one embodiment of the present invention, the hydrophilic agent is antitumor drug, protein drug, antibiotic or growth factor.

According to one embodiment of the present invention, the antitumor drug is doxorubicin (DOX) or cisplatin (CDDP).

According to one embodiment of the present invention, the bioactive agent in the protein shell is a hydrophobic agent.

According to one embodiment of the present invention, the hydrophobic agent is curcumin (Cur) or paclitaxel.

### BRIEF DESCRIPTION OF THE DRAWINGS

The invention can be more fully understood by reading the following detailed description of the embodiment, with reference made to the accompanying drawings as follows:

FIG. 1 illustrates a schematic cross-sectional view of a drug carrier according to an embodiment of the present invention.

FIG. 2 illustrates a schematic cross-sectional view of a drug carrier according to another embodiment of the present invention.

FIG. 3 illustrates a flow chart for making a drug carrier according to an embodiment of the present invention.

FIG. 4 illustrates a schematic cross-sectional view of a drug carrier according to an embodiment of the present invention.

FIG. 5 illustrates a schematic cross-sectional view of a drug carrier according to another embodiment of the present invention.

FIG. 6 illustrates a flow chart for making a drug carrier according to an embodiment of the present invention.

FIGS. 7A through 7C are electron microscope images of drug carriers according to an embodiment of the present invention.

FIGS. 8A through 8D illustrate particle size distribution diagrams of drug carriers with different graphene concentrations according to an embodiment of the present invention.

FIG. 9A illustrates a temperature vs. time graph of an embodiment and comparative examples under near infrared irradiation.

FIG. 9B illustrates a bar chart for drug-loading capacity of an embodiment and comparative examples.

FIGS. 9C and 9D illustrate cumulative release vs. time graphs of an embodiment and comparative examples under different near infrared irradiation durations and intensities.

FIGS. 10A and 10B illustrate cell viability vs. drug carrier concentration graphs of different cells after incubation with various concentrations of drug carriers according to an embodiment of the present invention.

FIGS. 11A through 11D are confocal microscope images of targeting tests for a drug carrier according to an embodiment of the present invention with different cells.

FIG. 11E is a confocal microscope image of a targeting test for a drug carrier according to an embodiment of the present invention.

FIG. 11F is a cross-sectional view along line A-A′ in FIG. 11E.

FIG. 11G is a cross-sectional view along line B-B′ in FIG. 11E.

FIGS. 12A through 12D are confocal microscope images of toxicity tests for photothermal therapy of an embodiment and comparative examples.

FIG. 12E illustrates a cell viability bar chart of embodiments and comparative examples under different conditions.

FIG. 13 is fluorescence images of a mouse injected with drug carriers having fluorescence labels after 1 day and 5 days.

FIG. 14 is infrared thermal images of a mouse injected with drug carriers and irradiated by near infrared after different durations.

FIG. 15A illustrates a relative tumor volume vs. time graph of mice after treated by different conditions.

FIG. 15B illustrates a mass vs. time graph of mice after treated by different conditions.

FIGS. 16A through 16C are pictures of mice and schematic graphs of growth status for tumor cells treated by different conditions.

FIGS. 17A through 17C illustrate schematic graphs of carriers under near infrared irradiation treatment.

FIG. 18 is an electron microscope image of drug carriers according to an embodiment of the present invention.

FIG. 19 illustrates a bar chart for iron concentration in tumor regions of embodiments and comparative examples.

FIG. 20 illustrates a cumulative release vs. time graph of a drug carrier under different environments according to an embodiment of the present invention.

FIG. 21 is an electron microscope image of drug carriers according to an embodiment of the present invention.

FIG. 22 is an electron microscope image of drug carriers according to another embodiment of the present invention.

FIG. 23 illustrates a nerve growth factor release vs. time graph of an embodiment and a comparative example.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples. Therefore, the spirit and scope of the appended claims should not be limited to the description of the embodiments contained herein.

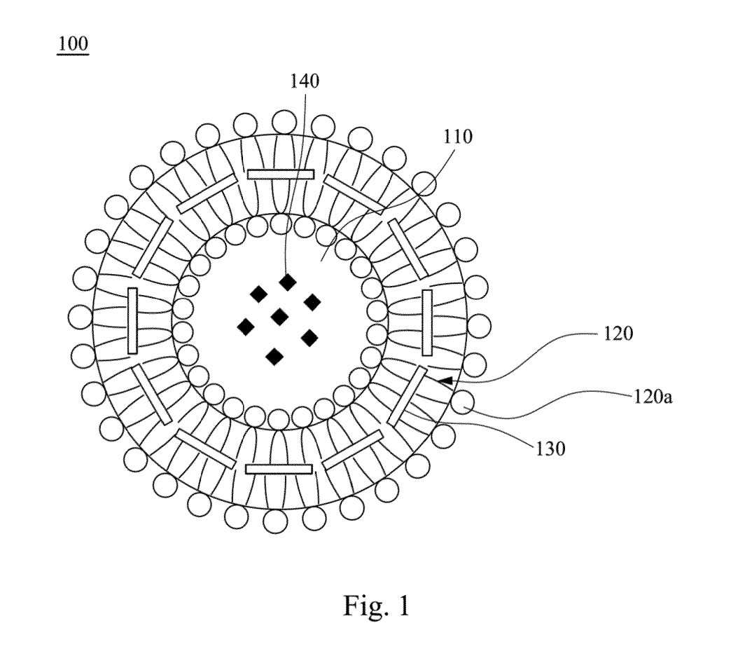

Referring to FIG. 1, which illustrates a schematic cross-sectional view of a drug carrier 100 according to an embodiment of the present invention. The drug carrier 100 includes an aqueous solution 110, a lactoferrin shell 120, graphenes 130, and a bioactive agent 140. The protein shell 120 includes a hydrophilic/hydrophobic layer, and encloses the aqueous solution 110. The graphenes 130 are dispersed in the protein shell 120, and the bioactive agent 140 is in the aqueous solution 110. The term “hydrophilic/hydrophobic” used herein means that the protein shell 120 has characteristics of both hydrophilic and hydrophobic.

It is noteworthy that the protein shell 120 illustrated in FIG. 1 includes one hydrophiliclhydrophobic layer. The hydrophilic/hydrophobic layer may be composed of two hydrophilic layers and one hydrophobic layer, and the structure of the hydrophilic/hydrophobic layer is stabilized by the hydrophobic layer. The two hydrophilic layers are the inner shell and the outer shell of the protein shell 120, and the hydrophobic layer is sandwiched by the two hydrophilic layers. As shown in FIG. 1, the hydrophilic layers are formed by hydrophilic heads of the proteins 120a, and the hydrophobic layer is formed by hydrophobic tails of the proteins 120a. In other embodiments, the protein shell of the drug carrier of the present invention includes more than one hydrophilic/hydrophobic layers, which the protein shell is composed of more than two hydrophilic layers and a plurality of hydrophobic layers, and each hydrophobic layer is sandwiched by each two hydrophilic layers.

The protein shell 120 is formed by self-assembly of protein 120a. The protein 120a may be amphiphilic lactoferrin, albumin or silk protein, and may have a concentration of about 1-5 wt % in the drug carrier 100.

The protein 120a can be recognized by the immune system of the body. Therefore, the drug carrier would not be attacked or swallowed by immune cells or proteins during delivery. Further, nanoparticles are known to elicit serum proteins as a coating when they are in circulation, which typically lead to enhanced uptake of nanoparticles by macrophages. As a result, nanoparticles are lost during delivery and no longer available to tumor. Currently, the most common approach to prevent this from happening is to use a polyethylene glycol (PEG) coating. Coating proteins appear on many natural nanoparticles, such as apolipoproteins on lipoproteins, which can also reduce the occurrence of uptake by macrophages. Therefore, by incorporating the protein 120a as a design coating in the drug carrier 100, the unintended serum protein coating can be prevented.

In some embodiments, the protein 120a is amphiphilic lactoferrin. In current technology, lactoferrin is rarely used as the structure of a carrier. Mostly, the lactoferrin is used as an enclosed drug for a therapeutic purpose, or grafted on a carrier surface for other purposes. The drug carrier of the present invention applies lactoferrin as the shell of the drug carrier, and because the lactoferrin is one of the components of the immune system of the body, it can be recognized by immune cells or proteins; therefore, the drug carrier would not be swallowed during delivery. The circulation time of the drug carrier of the present invention in the body can be improved due to the protection of the lactoferrin. Further, the lactoferrin has an ability to cross blood-brain barrier (BBB) and a function and specificity in tumor targeting, especially for brain tumor. Therefore, the drug carrier of the present invention exhibits considerable potential for treating brain tumor.

The graphenes 130 dispersed in the protein shell 120 is as a supporting structure to stabilize the drug carrier 100. In some embodiments, the graphenes 130 is dispersed in the hydrophobic layer of the protein shell 120.

In some embodiments, the graphenes 130 have a concentration of about 0.05-0.5 wt % in the drug carrier 100. When the concentration of the graphenes is reduced to about 0.02 wt %, the drug carrier will collapse, which is because the rigid graphenes cannot support the structure when the concentration is not sufficient. When the concentration of the graphenes is increased to about 0.6 wt %, the protein will deposit on the surface of the graphene in a manner of coating, and a sphere could not be formed. The concentration of the graphenes 130 has to be controlled in this range to form a stable carrier.

The graphenes 130 can absorb near infrared (NIR), and heat is generated in the graphenes 130 after the absorption of NIR, which causes the deformation or collapse of the drug carrier 100, and thereby the drug 140 enclosed by the protein shell 120 is released. Therefore, through applying NIR stimulation, the timing of the release of the bioactive agent 140 can be controlled, and the drug carrier achieves the effect of photothermal therapy. In some embodiments, the graphenes 130 are hydrophobic reduced graphene oxides modified from reduced graphene oxides. In some embodiments, the dimension of graphenes 130 is nanoscale, which the diameter is about 20-100 nm. Moreover, the graphene 130 is susceptible to electrical stimulation. Therefore, when applying external electrical field to the drug carrier 100, the graphene 130 is stimulated to vibrate, which leads to the structural destruction of the drug carrier 100, and the bioactive agent 140 is then released. The timing of the release of the bioactive agent 140 can be controlled through the intensity and duration of the electrical stimulation.

In current technology, although there are drug carriers using graphene, most of the carriers are formed by modification of a single sheet of graphene or by extra grating bioactive agent on graphenes. The preparing processes for these kinds of carriers are complicated, and the bioactive agent is only carried by a single plane of the graphenes, and thus small amount of bioactive agent can be loaded. The drug carrier of the present invention is a sphere formed by emulsion of protein and hydrophobic graphenes, and the bioactive agent is encapsulated inside the sphere. Comparing to planar graphene carrier, the drug carrier of the present provides three-dimensional space, and can load more amounts of the bioactive agent. Therefore, the drug carrier of the present invention can load more amounts of the bioactive agent without undergoing complicate modification or extra grafting.

The bioactive agent 140 is in the aqueous solution 110, and is a hydrophilic agent. In some embodiments, the bioactive agent 140 is an antitumor drug, such as doxorubicin (DOX) and cisplatin (CDDP). In other embodiments, the bioactive agent 140 is a growth factor, such as nerve growth factor or genipin.

Given the above, because the protein 120a in the drug carrier 100 is a protein in human body, the phagocytosis of the drug carrier by the immune system can be reduced, and the circulation time can be improved. Further, when the protein 120a is lactoferrin, the drug carrier 100 has targeting specificity in brain cells and an ability to cross blood-brain barrier. The graphenes 130 in the drug carrier 100 can stabilize the shell of the drug carrier 100, and by dual therapy of combining photothermal therapy and chemotherapy, only low dosage of bioactive agent is needed to achieve high-toxic capability, which, in clinical, the dosage of the bioactive agent can be decreased to reduce side effects, and to achieve the therapeutic effects caused by high dosage. Moreover, the amount and timing of the release of the bioactive agent can be controlled by different intensities and durations of NIR stimulation or electrical stimulation.

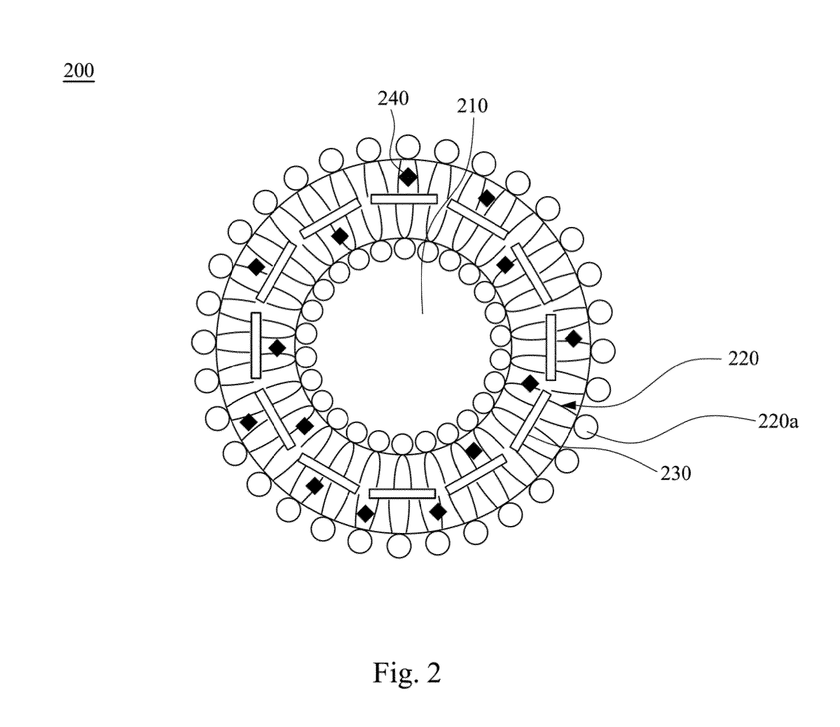

Referring to FIG. 2, which illustrates a schematic cross-sectional view of a drug carrier 200 according to another embodiment of the present invention. The drug carrier 200 includes an aqueous solution 210, a protein shell 220, graphenes 230, and a bioactive agent 240. The lactoferrin shell 220 is formed by self-assembly of protein 220a, and encloses the aqueous solution 210. The protein shell 220 includes a hydrophilic/hydrophobic layer. The graphenes 230 and the bioactive agent 240 are dispersed in the protein shell 220.

The bioactive agent 240 is in the protein shell 220. In some embodiments, the bioactive agent 240 is a hydrophobic agent, such as curcumin (Cur) and paclitaxel, and is in the hydrophobic layer of the protein shell 220.

The difference between the drug carrier 200 and the drug carrier 100 is that the bioactive agent 140 of the drug carrier 100 is in the aqueous solution 110, while the bioactive agent 240 of the drug carrier 200 is in the protein shell 220. This difference does not affect the functions of each component in the embodiment, such as the protein 220a and the graphenes 230. Therefore, the drug carrier 200 has the same functions and advantages as the drug carrier 100.

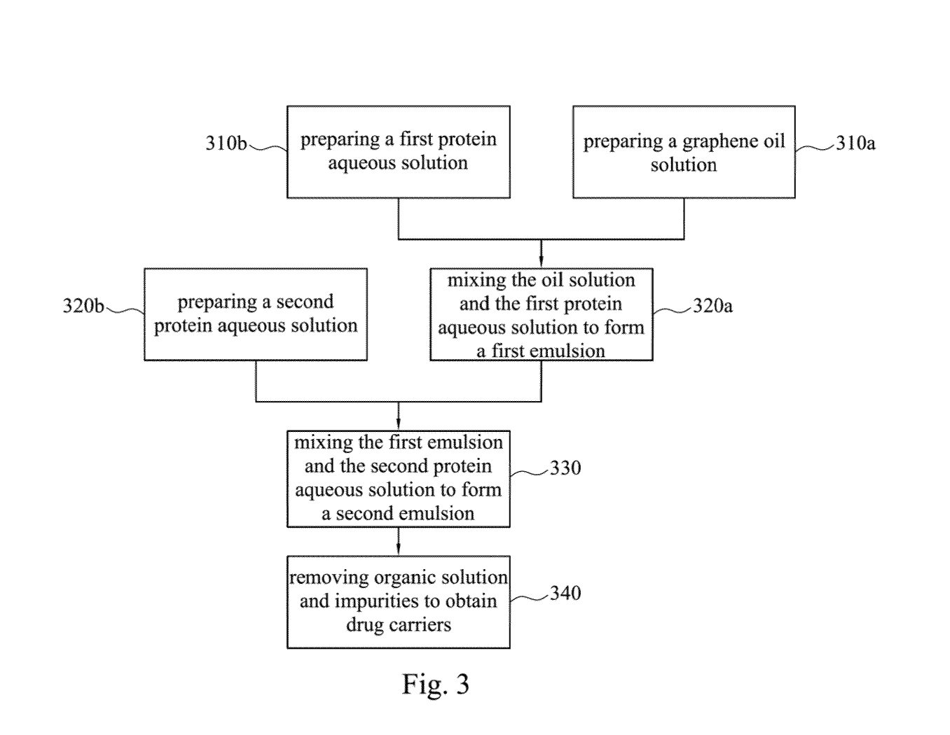

Referring to FIG. 3, which illustrates a flow chart for making the drug carrier 100 or the drug carrier 200 according to an embodiment of the present invention. First, a graphene oil solution 310a and a first protein aqueous solution 310b are prepared respectively, and the oil solution and the first protein aqueous solution are mixed and emulsified to form a water-in-oil (w/o) type first emulsion 320a. Next, a second protein aqueous solution is prepared 320b, and the first emulsion and the second protein aqueous solution are mixed and emulsified to form a water-in-oil-in-water (wlo/w) type second emulsion 330. Then, the organic solution and impurities in the second emulsion are removed to obtain drug carriers 340.

The bioactive agent 140 loaded in the drug carrier 100 is a hydrophilic agent, and the method for making the drug carrier 100 includes dissolving the bioactive agent 140 in the first protein aqueous solution in step 310b to be encapsulated in the drug carrier 100 by the aqueous solution. The bioactive agent 240 loaded in the drug carrier 200 is a hydrophobic agent, and the method for making the drug carrier 200 includes dissolving the bioactive agent 240 in the graphene oil solution in step 310a to be encapsulated in the drug carrier 200 by the oil solution.

In step 320a, mixing the oil solution and the first protein aqueous solution to form the first emulsion includes mixing, emulsifying, and stirring. In this step, aqueous hollow carriers are formed by self-assembly of the protein, and are dispersed in the oil solution to obtain the water-in-oil type first emulsion.

In step 340, removing the organic solution and impurities in the second emulsion includes evaporating the organic solution, and centrifuging and washing to remove the impurities. The impurities include unreacted materials.

In some embodiments, the protein is amphiphilic lactoferrin, which include hydrophobic tails and a hydrophilic head, and thus the amphiphilic lactoferrin and the hydrophobic graphenes are arranged to form the hollow carriers based on the hydrophobic and hydrophilic structures during emulsion. This process does not require the assistance of other emulsifiers, and the drug carriers can be made simply by emulsifying steps.

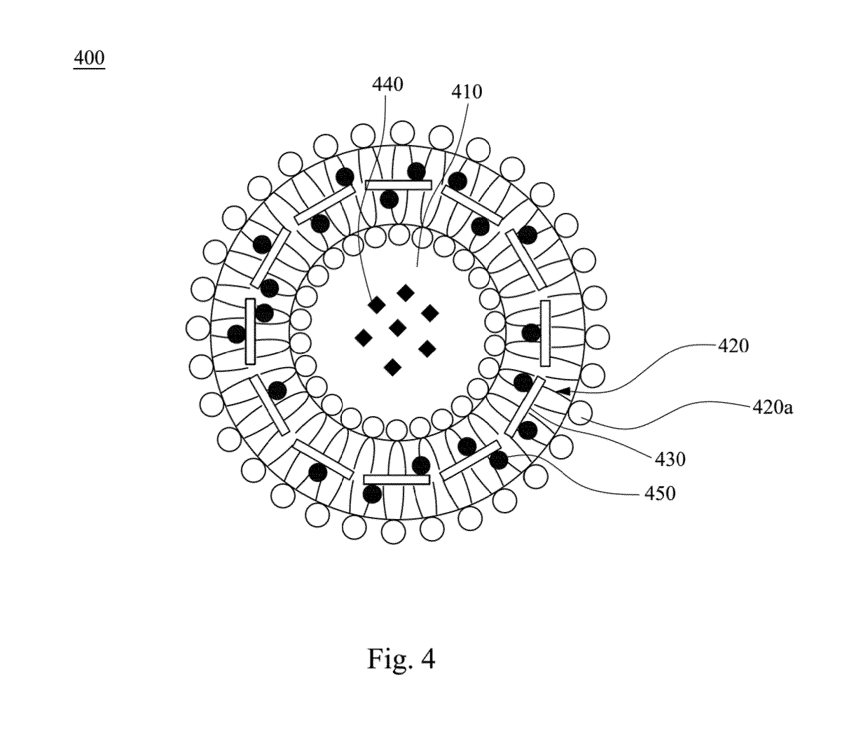

Referring to FIG. 4, which illustrates a schematic cross-sectional view of a drug carrier 400 according to an embodiment of the present invention. The drug carrier 400 includes an aqueous solution 410, a protein shell 420, graphenes 430, a bioactive agent 440, and iron oxide 450. The protein shell 420 is formed by self-assembly of protein 420a, and encloses the aqueous solution 410. The protein shell 420 includes a hydrophilic/hydrophobic layer. The graphenes 430 and the iron oxide 450 are dispersed in the protein shell 420, and the bioactive agent 440 is in the aqueous solution 410.

In some embodiments, the iron oxide 450 is iron(II,III) oxide nanoparticle with formula Fe₃O₄, and is in the hydrophobic layer of the protein shell 420.

Because the drug carrier 400 includes iron oxide 450, the drug carrier 400 has functions of imaging and magnetic guide control. For instance, the behavior of the iron oxide 450 in the body can be controlled by magnets or magnetic fields. Therefore, in addition to photothermal therapy, the drug carrier 400 has a function of magnetic guide. Guided by a magnet, the brain targeting for the drug carrier 400 can be improved; and further, the drug carrier 400 can be guided to other designated therapeutic areas. Therefore, the cumulative amount of the drug carrier 400 in the designated therapeutic area can be increased. Moreover, with the iron oxide 450, the drug carrier 400 can have a capability of being imaged by magnetic resonance imaging (MRI).

The difference between the drug carrier 400 and the drug carrier 100 is that the drug carrier 400 further includes the iron oxide 450 comparing to the drug carrier 100. This difference does not affect the functions of components other than the iron oxide 450 in the embodiment, such as the protein 420a and the graphenes 430. Therefore, the other components of the drug carrier 400 have the same functions and advantages as the drug carrier 100.

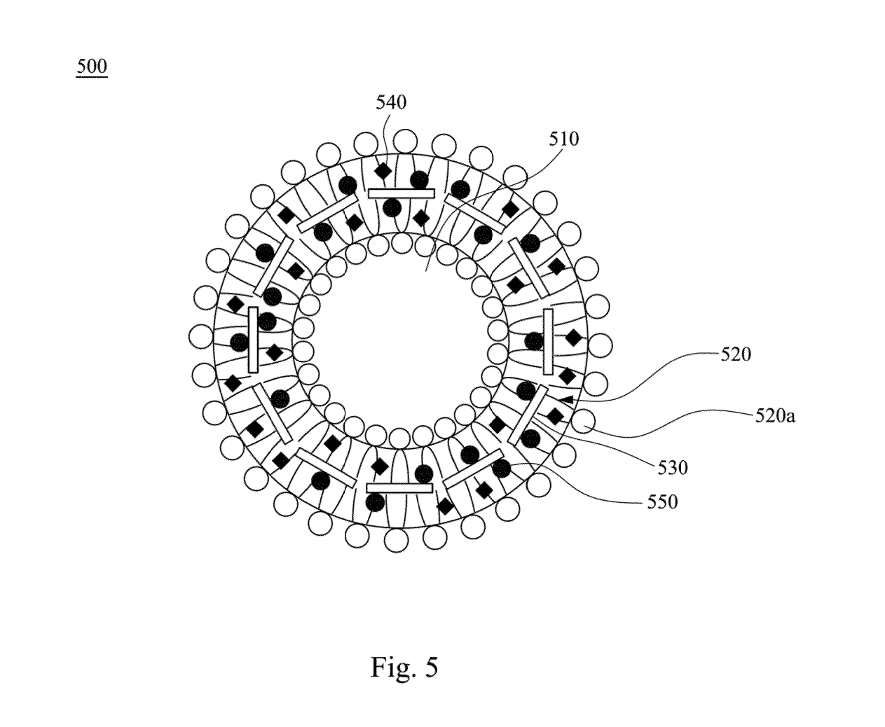

Referring to FIG. 5, which illustrates a schematic cross-sectional view of a drug carrier 500 according to another embodiment of the present invention. The drug carrier 500 includes an aqueous solution 510, a protein shell 520, graphenes 530, a bioactive agent 540, and iron oxide 550. The protein shell 520 is formed by self-assembly of protein 520a, and encloses the aqueous solution 510. The protein shell 520 includes a hydrophilic/hydrophobic layer. The graphenes 530, the bioactive agent 540 and the iron oxide 550 are dispersed in the protein shell 520. In some embodiments, the graphenes 530, the bioactive agent 540 and the iron oxide 550 are dispersed in the hydrophobic layer of the protein shell 520.

The difference between the drug carrier 500 and the drug carrier 400 is that the bioactive agent 440 of the drug carrier 400 is in the aqueous solution 410, while the bioactive agent 540 of the drug carrier 500 is in the protein shell. This difference does not affect the functions of each component in the embodiment, such as the lactoferrin 520a, the graphenes 530, and the iron oxide 550. Therefore, the drug carrier 500 has the same functions and advantages as the drug carrier 400.

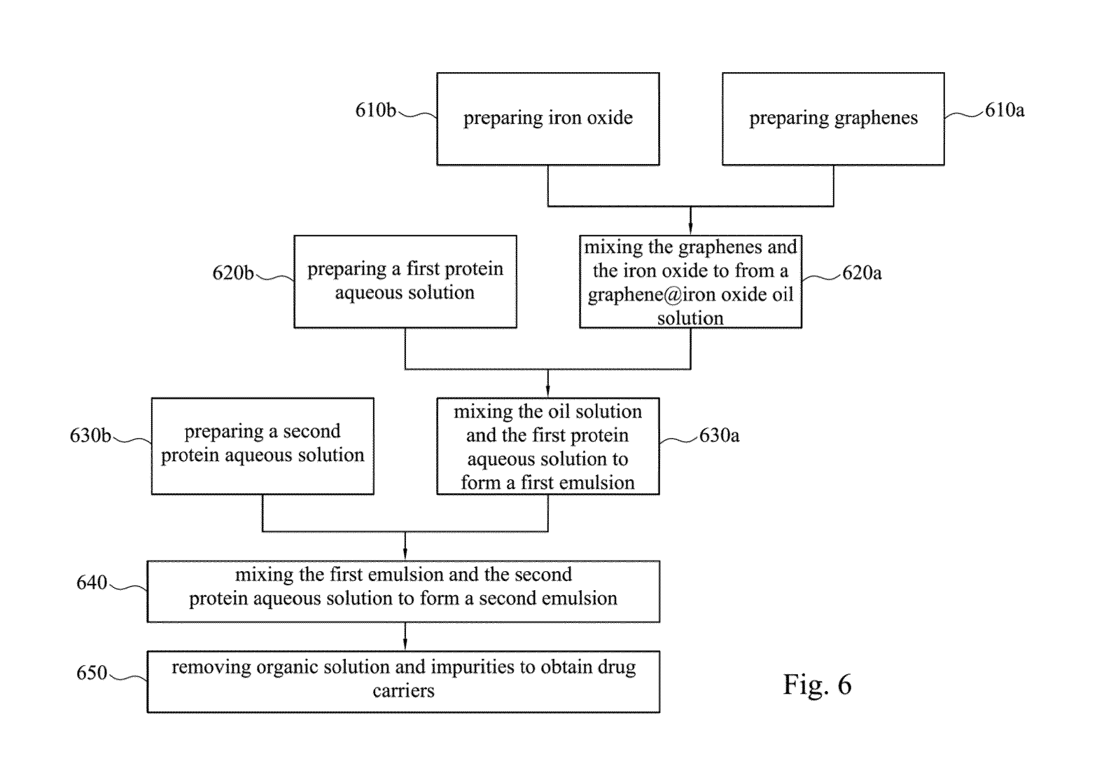

Referring to FIG. 6, which illustrates a flow chart for making the drug carrier 400 or the drug carrier 500 according to an embodiment of the present invention. First, graphenes 610a and iron oxide 610b are prepared respectively, and the graphenes and the iron oxide are mixed to form an oil solution having the graphenes the iron oxide (Graphene@iron oxide oil solution). Next, a first protein aqueous solution is prepared 620b, and mixed and emulsified with the graphene@iron oxide oil solution to form a water-in-oil (w/o) type first emulsion 630a. A second protein aqueous solution is prepared 630b, and the first emulsion and the second protein aqueous solution are mixed and emulsified to form a water-in-oil-in-water (w/o/w) type second emulsion 640. Then, the organic solution and impurities in the second emulsion are removed to obtain drug carriers 650.

In step 610b, the iron oxide is dissolved in an oil solution in a concentration of 5-30 mg/mL. The concentration of the iron oxide has to be controlled in this range to form spherical shaped carriers.

The bioactive agent 440 loaded in the drug carrier 400 is a hydrophilic agent, and the method for making the drug carrier 400 includes dissolving the bioactive agent 440 in the first protein aqueous solution in step 620b to be encapsulated in the drug carrier 400 by the aqueous solution. The bioactive agent 540 loaded in the drug carrier 500 is a hydrophobic agent, and the method for making the drug carrier 500 includes dissolving the bioactive agent 540 in the graphene@iron oxide oil solution in step 620a to be encapsulated in the drug carrier 500 by the oil solution.

In step 630a, mixing the oil solution and the first protein aqueous solution to form the first emulsion includes mixing, emulsifying, and stirring. In this step, aqueous hollow carriers are formed by self-assembly of the protein, and are dispersed in the oil solution to obtain the water-in-oil type first emulsion.

In step 650, removing the organic solution and impurities in the second emulsion includes evaporating the organic solution, and centrifuging and washing to remove the impurities. The impurities include unreacted materials.

In some embodiments, the making process applies amphiphilic lactoferrin, and hydrophobic reduced graphene oxide and hydrophobic iron oxide as the oil solution. The amphiphilic lactoferrin, the hydrophobic graphenes, and hydrophobic iron oxide are arranged to form the hollow carriers based on the hydrophobic and hydrophilic structures during emulsion. This process does not require the assistance of other emulsifiers. The drug carriers made by this process has additional functions of imaging and magnetic guide control because of encapsulating the iron oxide.

It is noteworthy that the drug carrier of the present invention may include the bioactive agent in the aqueous solution or the protein shell, or may include the bioactive agents in both of the aqueous solution and the protein shell. The bioactive agents in the aqueous solution and the protein shell may be the same or different.

The drug carrier of the present invention combines protein and graphene, and is made simply by emulsifying steps. The drug carrier is a multifunctional nanocomposite hollow drug carrier with protein as the main structure, which the diameter of the drug carrier can be adjusted by the concentration of the protein, and can effectively encapsulate the bioactive agent and reduce the toxicity of the bioactive agent. When the drug carrier of the present invention is used to encapsulate a cancer drug, the side effects of the cancer drug can be reduced. Conventional polymer carrier has to be modified or grafted with other molecules to reduce the phagocytosis of the polymer carrier by the immune cells or proteins. Unlike the conventional polymer carrier, the drug carrier of the present invention includes the protein recognized by the immune system, and can increase the circulation time of the drug carrier in the body to arrive at the designated therapeutic area. Further, the graphene can absorb the NIR; therefore, the timing of drug release can be controlled, and the drug carrier has an effect of photothermal therapy. Moreover, the drug carrier of the present invention may further include iron oxide, and in addition to the photothermal therapy effect, the drug carrier of the present invention has a function of magnetic guide. By magnetic guide, the drug carrier can be guided to the designated therapeutic area, and the targeting of the drug carrier for this area can be enhanced. The drug carrier of the present invention is a non-toxic protein nanocomposite hollow carrier that is made by simple process and has multiple functions. Other than cancer treatment, the drug carrier can achieve other therapeutic purposes by altering the materials encapsulated therein, such as tissue repairing by encapsulating DNA or other repairing proteins, and bioimaging by encapsulating fluorescent materials, and has great potential for clinical medicine.

The detailed description provided below is intended as a description of the present examples and is not intended to represent the only forms in which the present example may be constructed or utilized. Therefore, the spirit and scope of the appended claims should not be limited to the description of the embodiments contained herein.

### Synthesis of Reduced Graphene Oxide

Nanoscale reduced graphene oxide (rGO) is synthesized by a modified Hummer method, which includes the following steps:
1. Native graphite flakes were mixed with H₂SO₄, K₂S₂O₈, and P₂O₅, and heated at 80° C. for 12 hours.
2. The graphite flake of step 1 were washed by distilled water and ethanol, and dried overnight in an environment with nitrogen to form graphite powders.
3. The dried graphite powders were added into H₂SO₄, and KMnO₄ was added slowly with stirring to oxidize the graphite powders.
4. The mixture of step 3 was continuously stirred in an ice bath for 2 hours. Then, the reaction was terminated with distilled water.
5. To remove MnO₂, H₂O₂ was added to the mixture of step 4 until the color of the mixture changed to bright yellow.
6. 1% HCl was added to the mixture of step 5, and followed by centrifuging at 6000 rpm for 10 minutes, and washing with distilled water for 3 times until the pH reached 6-7. The graphite oxide powders were then purified.
7. The oxidized graphite was exfoliated by an ultrasonic probe, and centrifuged at 12000 rpm for 30 minutes to collect small-size and uniform graphene oxide (GO).
8. The graphene oxide was added to dilute ammonia solution to adjust the pH to 11.5-11.8, and heated at 80° C. for 12 hours. After the reaction, the color of the solution turned from brown to black.
9. The solution of step 8 was sonicated for about 1 hour to form uniform rGO dispersion. The dispersion was centrifuged at 12000 rpm for 30 minutes to remove large-size rGO sheets, and small-size rGO sheets were then obtained.

### Preparation of Hydrophobic Reduced Graphene Oxide

The process for modifying the above nanoscale rGO sheets to become hydrophobic includes the following steps:
1. 100 g of rGO was mixed with 400 mg of aliphatic amine, and the mixture is dissolved in 100 mL of ethanol and reacted at room temperature for 2 hours.
2. The hydrophobic rGO was separated by filtration using a nylon membrane with pore size of 0.2 μm and washed by ethanol for several times to remove excess aliphatic amine.

### Preparation of Drug Carriers

According to an embodiment of the present invention, the method for preparing drug carriers, which the protein shell is made of lactoferrin, includes the following steps:
1. 0.75 mg of hydrophobic rGO was dispersed in 250 μL of chloroform as an oil solution.
2. 2 mg of lactoferrin (Lf) and 0.5 mg of doxorubicin (DOX) were dissolved in 100 μL of de-ionized water as a first aqueous solution.
3. The first aqueous solution of step 2 was mixed with the oil solution of step 1, and emulsified by ultrasonifucation (20 kHz, 130 W) for 1 minute to form a water-in-oil (w/o) first emulsion.
4. 400 μL of 2% lactoferrin was prepared as a second aqueous solution.
5. The second aqueous solution of step 4 was emulsified with the first emulsion of step 3 using similar process described in step 3 to form a water-in-oil-in-water (w/o/w) second emulsion.
6. The second emulsion of step 5 was stirred at room temperature until the organic solvent was completely evaporated.
7. The products of step 6 were centrifuged to remove unreacted materials, and were dispersed in double-distilled water to obtain the drug carriers. The structure of the drug carriers may refer to the drug carrier 100 shown in FIG. 1.

The drug carriers obtained from the abovementioned method were used in the experiments of the following Experimental examples 1-8. It is noteworthy that a “non-drug loaded carrier” in the present specification represents a carrier having a structure similar to the drug carrier 100, but the aqueous solution of the carrier does not include a drug.

### Experimental Example 1

In order to make the drug carrier of the present invention successfully, the diameter of the rGO has to be controlled in a range of 20-100 nm. A scanning electron microscope (SEM) and a transmission electron microscope (TEM) were used in this experimental example to examine the morphology and size of the drug carrier.

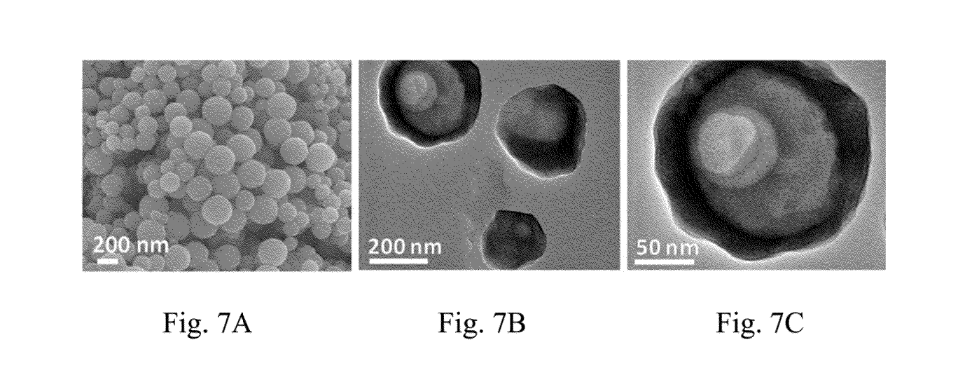

Referring to FIGS. 7A through 7C, which shows electron microscope images of drug carriers according to an embodiment of the present invention. FIG. 7A is a SEM image, FIG. 7B is a TEM image, and FIG. 7C is a partial enlargement view of FIG. 7B. As shown in FIG. 7A, the diameter of the drug carriers was about 100-350 nm, and no obvious crevices or cracks were observed from the surfaces of the drug carriers, which means that the structure composed of lactoferrin and graphenes was perfect and stable.

As shown in FIGS. 7B and 7C, the drug carriers had a double shell structure with a hollow core. The shell thickness was about 25 nm, and no clear phase separation existed between two shells. The contrast of the inorganic rGO in the hydrophobic layer is stronger, and therefore the hydrophobic layer is darker than the inner and outer hydrophilic layers of the organic lactoferrin shell. The lactoferrin shell contains rGO, which makes the carrier has a stable structure. Although there is literature suggest that lactoferrin can be used as a surfactant, the lactoferrin cannot be emulsified alone. If there is only lactoferrin and chloroform without the presence of rGO, no particles or spheres would be formed, and only membrane would be formed. A carrier would only be formed in the presence of the rGO.

### Experimental Example 2

In the drug carrier of the present invention, the rGO plays roles of stabilizing the structure of the carrier, absorbing near infrared (NIR) to achieve the effect of photothermal therapy, and a factor of drug release induced by NIR. The concentration of the rGO has direct effects on the particle size and the synthesis or not of the lactoferrin/rGO drug carrier.

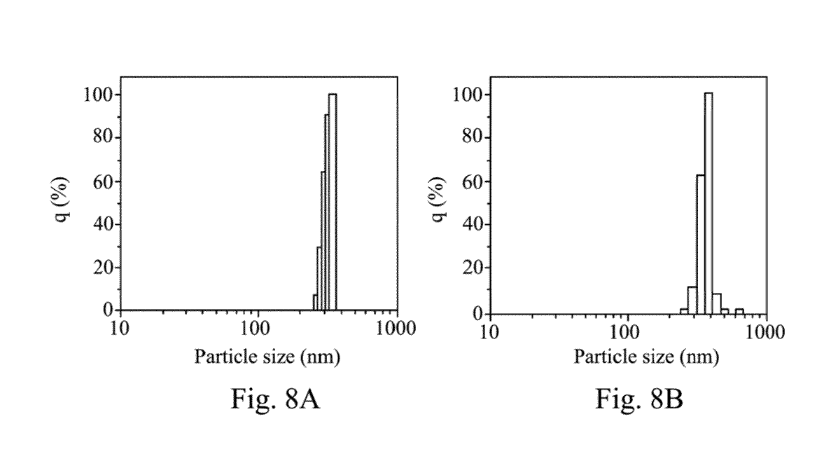

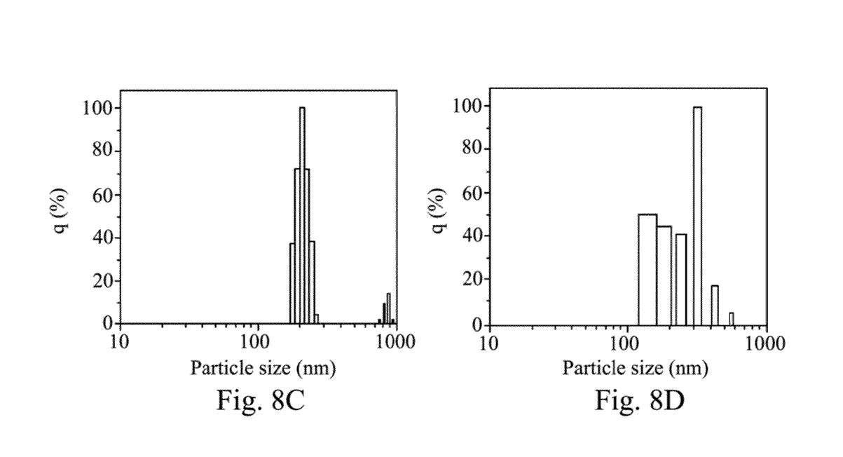

Referring to FIGS. 8A through 8D, which are particle size distribution diagrams of drug carriers with different graphene concentrations according to an embodiment of the present invention. FIGS. 8A through 8D show the particle size distribution diagrams, which were measured and analyzed by dynamic light scattering (DLS), of the drug carriers with graphene concentrations of 0.03 wt %, 0.15 wt %, 0.3 wt %, and 0.6 wt %, respectively. Referring to FIG. 8A, when the graphene concentration was 0.03 wt %, the carriers would form spheres after drying. However, the morphology of the carriers would collapse, and cannot be used as drug carriers. This is because the amount of the rigid rGO was not enough to support the structure of the carriers. Referring to FIGS. 8B and 8C, when the graphene concentration was 0.15 wt % and 0.3 wt %, the carriers had stable structures, and the particle sizes were in a range of 120-450 nm. Referring to FIG. 8D, when the graphene concentration was increased to 0.6 wt %, the lactoferin would coat on the surface of the graphenes, i.e. Lf-coated rGO, and no spheres would be formed. The particle sizes shown in the particle size distribution diagram included some rGO nanosheets and Lf-coated rGO. Therefore, only when the rGO is maintained at a suitable concentration, which is about 0.05-0.5 wt % would the carriers with stable structure be formed.

### Experimental Example 3

Experimental example 3 was to discuss the effects of NIR on the drug carrier of the present invention and the drug-loading capacity thereof.

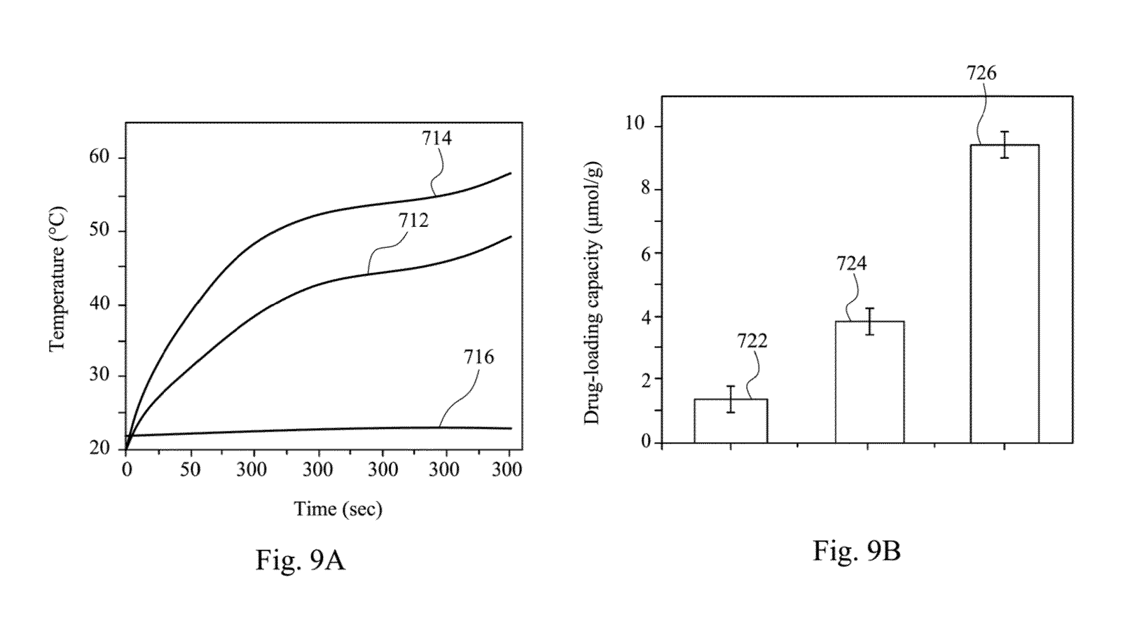

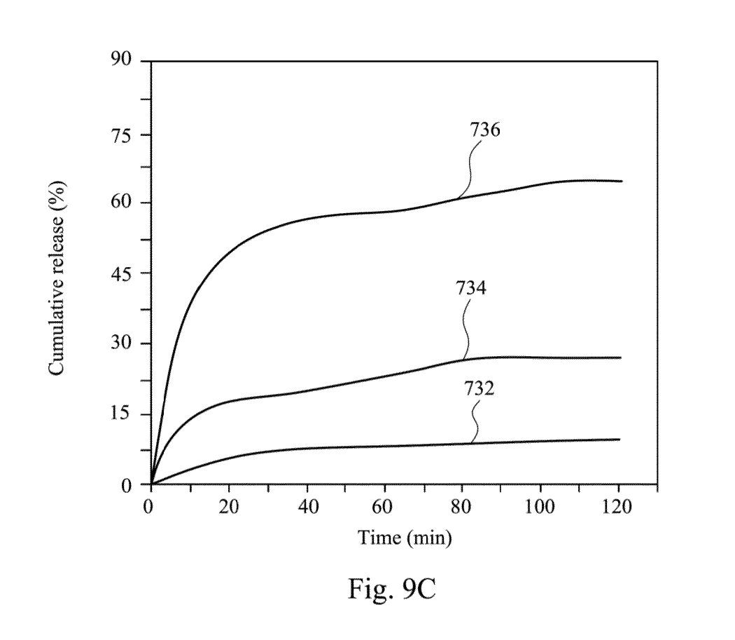

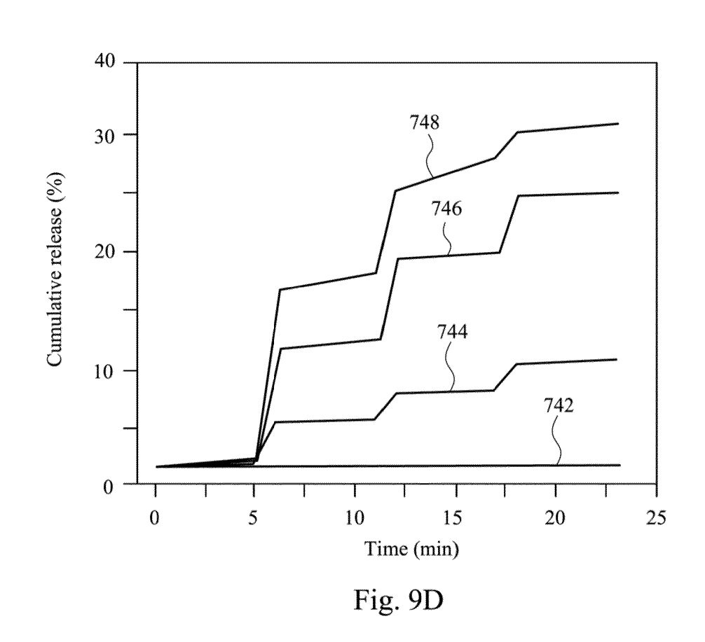

Because the rGO in the drug carrier of the present invention can absorb NIR and generate heat, the increase in temperature can be observed in both rGO and carriers after irradiated by NIR. Referring to FIG. 9A, which show a temperature vs. time graph of an embodiment and comparative examples dispersed in phosphate buffered saline (PBS), and irradiated by NIR (808 nm, 2 W/cm²). Line 712 represents the embodiment of carriers with lactoferrin and rGO, line 714 represents a comparative example of pure rGO, and line 716 represents another comparative example of pure lactoferrin. As shown in FIG. 9A, when the pure lactoferrin solution, i.e. line 716, was irradiated by NIR, there was no obvious changes in temperature, while the temperatures of both the pure rGO, i.e. line 714, and the carrier of the embodiment, i.e. line 712, showed obvious increases. Among which, the temperature increase in the carrier of the embodiment, i.e. line 712, was slight lower than that in the pure rGO of the comparative example, i.e. line 714, because the thermal conductivity of the lactoferrin is poorer than that of the rGO. Nevertheless, the carriers of the embodiment still achieved more than 85% absorption efficiency for NIR comparing to the pure rGO of the comparative example, and thus would not have much impact on the photothermal therapy effect.

Referring to FIG. 9B, which shows a bar chart for drug-loading capacity of an embodiment of the present invention and comparative examples. Line 722 represents pure rGO of a comparative example, line 724 represents rGO with lactoferrin coated thereon, i.e. Lf-coated rGO, of another comparative example, and line 726 represents the embodiment of carriers with lactoferrin and rGO. The drug used to test the drug loading capacity was DOX. Neither of the two comparative examples can form spheres, and the drug was loaded on the rGO sheets instead of being encapsulated. Therefore, in regard to the capability of drug-loading, because the aqueous core of the double-emulsion carrier of the embodiment provides a larger space to encapsulate aqueous drug, DOX, and leakage of the drug is prevented, the drug-loading capacity for the drug carrier of the embodiment (9.43 μmol/g) was far better than that of the rGO (1.35 μmol/g) and Lf-coated rGO (3.81 μmol/g) of the comparative examples.

FIGS. 9C and 9D illustrate cumulative release vs. time graphs of an embodiment and comparative examples under different NIR irradiation durations and intensities. Referring to FIG. 9C, line 732 represents drug carriers without NIR irradiation, and lines 734 and 736 represent drug carriers underwent NIR irradiation for 1 minute and 5 minutes respectively. The natural cumulative release of the drug carriers without NIR irradiation was less than 8% within 2 hours due to their stable structures. However, under the stimulation of NIR, partial heating occurs through the photothermal conversion of the rGO, and results in deformation of the shell structure or molecular arrangement if the drug carrier. When the drug carriers were irradiated by NIR for 1 minute and 5 minutes respectively, high cumulative release of 20% and 60% were achieved after 1 hour.

The drug carrier of the present invention has an ability of deformation recovery, but the ability depends on the stimulation intensity. When the stimulation intensity is too strong, the carrier would be destroyed and could not recover. Hence, when the stimulation is over 5 minutes, even though the NIR stimulation was removed, the unrecoverable deformation would still maintained at a rapid sustained drug release rate. Referring to FIG. 9D, when intermittent NIR stimulation was applied, which the frequency was about 1 minute, the drug release of the drug carriers followed a staircase-shaped profile, and the release dosage depended on the NIR intensity, suggesting that the thermally-induced release was an on-demand yet stoppable process. In FIG. 9D, NIR intensities of 0 W/cm², 0.4 W/cm², 0.8 W/cm², and 2 W/cm² were used to conduct experiments, the cumulative release of the drug over time of which were represented by lines 742, 744, 746, and 748, respectively. Similarly, the partial heating enhanced the permeability of the drug carrier shells and the drug diffusion rate. Removing the NIR stimulation within 5 minutes made the drug carriers return to the original states with negligible drug release. Such protein recovery process can be understood by peptide thermosensitive behavior, such as that the conformation of some peptides is like a free coil molecule in warm water, while turns into a gel structure with a triple-helix sequence in cool water. The drug release mode of the drug carrier of the present invention is thermosensitive and reversible, which can prevent natural drug release, and the drug release behavior can be manipulated precisely through tuning the NIR irradiation duration.

### Experimental Example 4

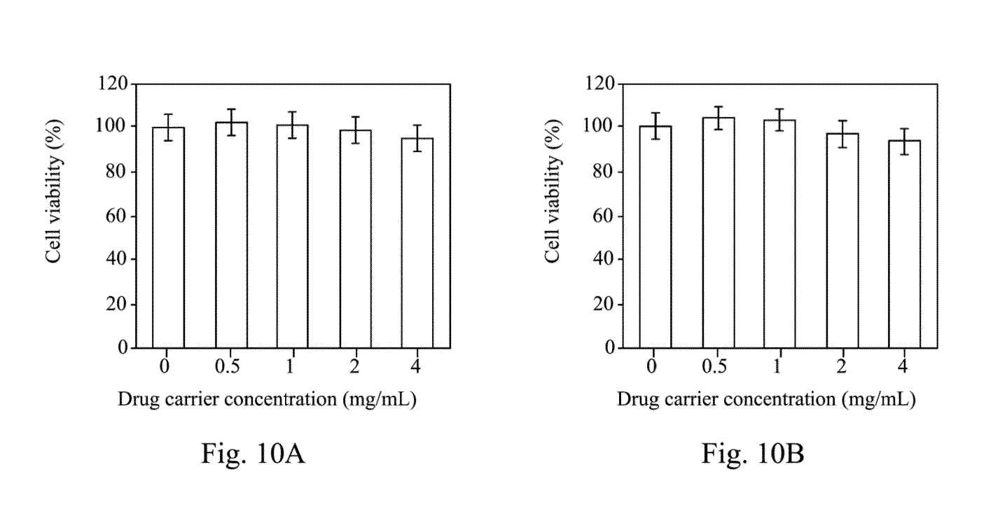

Biological safety test: The drug carrier of the present invention exhibits good biological safety and an ability of targeting specific tumor cells. In cell culture experiments, RG2 cell, which is a brain cancer cell line and has many lactoferrin receptors, and MRC-5 cell, which is a human lung fibroblast normal cell, and has few lactoferrin receptors, were used to conduct experiments for the biological safety test. Further, in order to test the cytotoxicity of the drug carrier of the present invention itself, the drug carriers used in Experimental example 4 did not include drug. Referring to FIGS. 10A and 10B, which are cell viability vs. drug carrier concentration graphs of RG2 cell and MRC-5 cell after incubation with various concentrations of drug carriers according to an embodiment of the present invention for 24 hours respectively. As shown in FIGS. 10A and 10B, even though the drug carrier concentration was up to 4 mg/mL, the cell viability still reached 93%±6%. Hence, the drug carrier of the present invention shows no toxicity and good biological safety to cells.

### Experimental Example 5

Targeting test: Drug carriers encapsulating fluorescent materials were detected by a flow cytometry to test that whether the drug carrier of the present invention can delivery drugs to tumor cells accurately. In this experimental example, the drug carriers encapsulating fluorescent materials were independently incubated with RG2 cells and MRC-5 cells for 4 hours, and the cells were stained and observed by a confocal microscope.

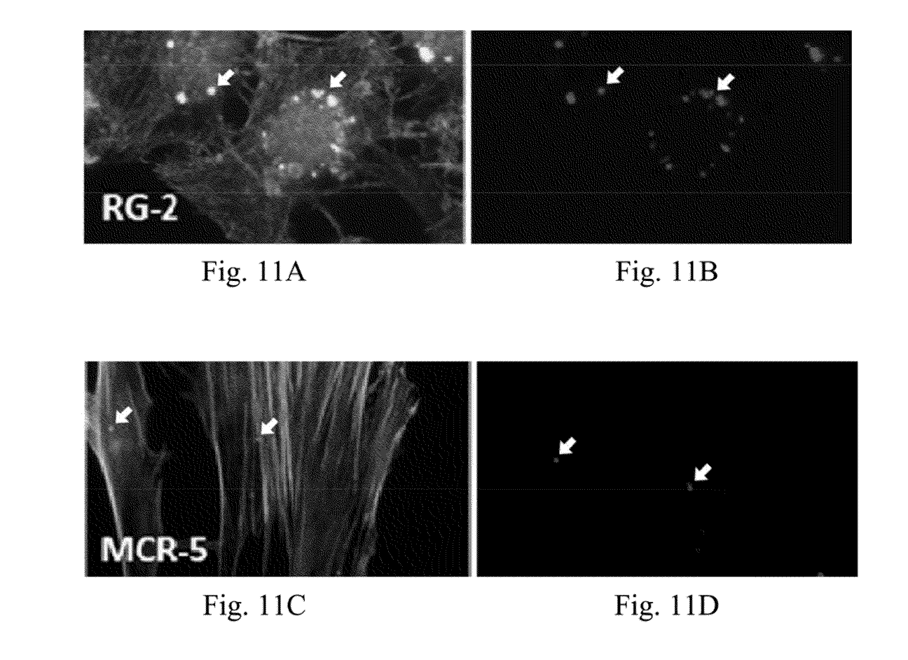

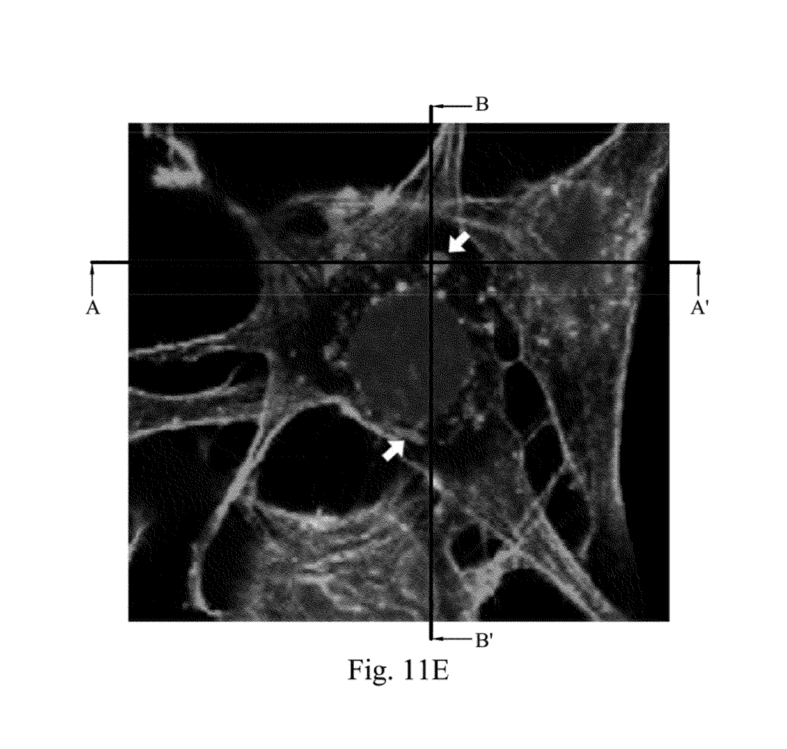

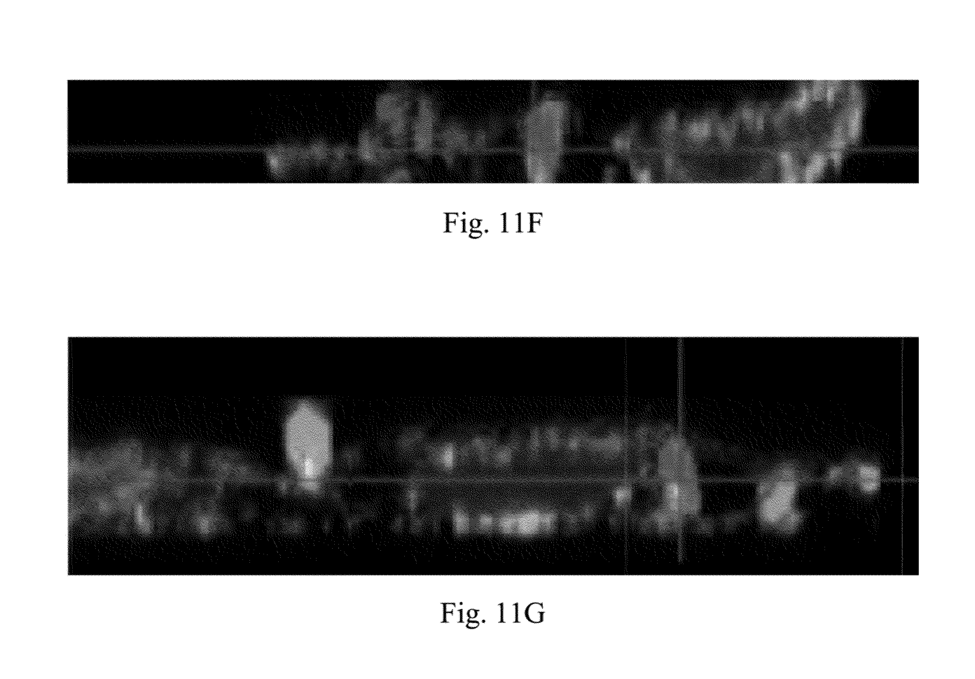

Referring to FIGS. 11A through 11D, which are confocal microscope images of targeting tests for the drug carriers of the present invention with different cells. FIGS. 11A and 11B shows the testing results with the RG2 cells, and FIGS. 11C and 11D shows the testing results with the MRC-5 cells. FIGS. 11A and 11C are combined confocal microscope images of cell staining images and fluorescent images of the drug carriers, while FIGS. 11B and 11D only shows the confocal microscope images of the fluorescent images of the drug carriers. Some of the drug carriers are indicated by arrows in FIGS. 11A through 11D.

As shown in FIGS. 11A and 11B, the drug carriers swallowed by the RG2 cells were observed in many regions within the cells, including the cytoplasm, and most of the drug carriers surrounded the nuclei. However, as shown in FIGS. 11C and 11D, only few drug carriers were observed at the cell surfaces, and did not be swallowed by the MCR-5 cells. The drug carrier of the present invention shows high targeting efficacy and internalized ability toward the RG2 cell. Further, the swallowed amount of the drug carriers showed great differences between the RG2 cells and the MCR-5 cells after incubating for 1 hour, suggesting that besides high targeting efficacy of the lactoferrin toward the RG2 cell, the drug carrier has an ability of passing through blood-brain barrier (BBB). Therefore, the drug carrier of the present invention shows great potential for treating brain cancer.

FIG. 11E shows a confocal microscope image of a targeting test for drug carriers of the present invention with RG2 cells, and some of the drug carriers are indicated by arrows. FIG. 11F is a cross-sectional view along line A-A′ in FIG. 11E, and FIG. 11G is a cross-sectional view along line B-B′ in FIG. 11E. As shown in FIGS. 11E through 11G, the drug carrier of the present invention can be swallowed by the RG2 cell and stays in many regions within the cell.

### Experimental Example 6

Toxicity tests for photothermal therapy: No carriers, non-drug loaded carriers, and drug carriers of the present invention with different DOX concentrations were independently incubated with RG2 cells in vitro for 1 hour, and irradiated by NIR for 5 minutes (808 nm, 2 W/cm²). Then, the cells were stained by propidium iodide and observed by a confocal microscope, and the cell viabilities were calculated.

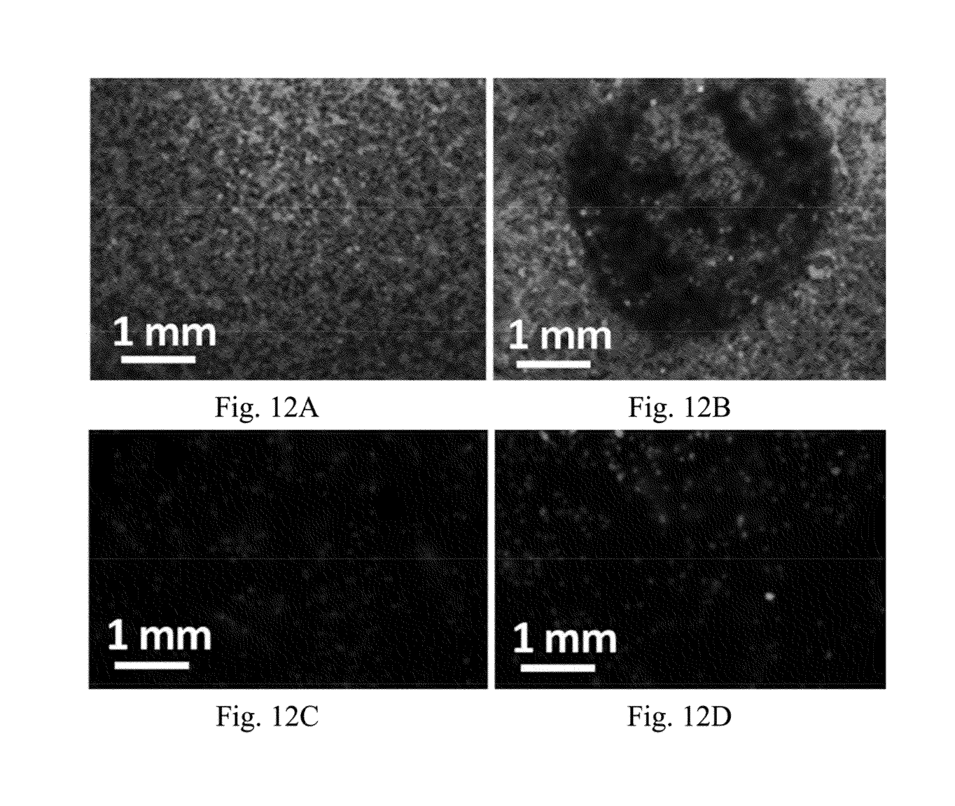

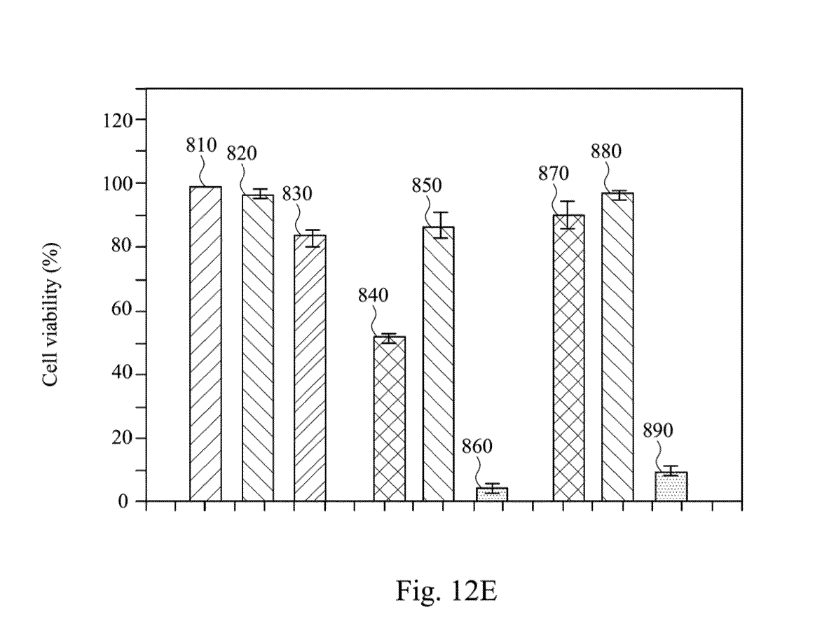

Referring to FIGS. 12A through 12D, which are confocal microscope images of RG2 cells being incubated with no carriers as a control, non-drug loaded carriers, and drug carriers with DOX concentrations of 0.25 μg/mL and 0.05 μg/mL after irradiated by NIR and stained, respectively. Referring to FIG. 12A, there was no cell killed for the control without carriers after the cell culture was irradiated by NIR for 4 minutes, which suggests that the NIR radiation alone would not kill the cells. Referring to FIG. 12B, when the cell culture incubated with the non-drug loaded carriers was irradiated by NIR, most of the cells were killed at the irradiation area, which is the darker portion in FIG. 12B, while in the area out of the NIR irradiation, most cancer cells still survived, which is the brighter portion in FIG. 12B. Referring to FIG. 12C, when the drug carrier with the DOX concentration of 0.25 μg/mL were used to incubated with the cells together with the NIR irradiation, not only the cells at the irradiation area, but also the cells in the area out of the irradiation were killed. Referring to FIG. 12D, when the drug-loading capacity was 0.2 times of the drug carrier used in FIG. 12C, most of the cancer cells were still killed with the cooperation of the photothermal therapy.

Referring to FIG. 12E, which is a cell viability bar chart of embodiments and comparative examples under different conditions after incubating with RG2 cells. The conditions for bars 810, 820, and 830 were without using of DOX, and bars 810, 820, and 830 represents the cell viabilities of no carriers, which is as a control, carriers without the NIR irradiation, and carriers with the NIR irradiation, respectively. The conditions for bars 840, 850, and 860 were using of 0.25 μg/mL DOX, and bars 840, 850, and 860 represents the cell viabilities of pure DOX, which is as a control, carriers without the NIR irradiation, and carriers with the NIR irradiation, respectively. The conditions for bars 870, 880, and 890 were using of 0.05 μg/mL DOX, and bars 870, 880, and 890 represents the cell viabilities of pure DOX, which is as a control, carriers without the NIR irradiation, and carriers with the NIR irradiation, respectively. As shown in FIG. 12E, because there were no DOX for bars 810, 820, and 830, few cells were killed. Bar 840 represents the control of pure DOX with the concentration of 0.25 μg/mL, which led about 50% of cells to death. Therefore, the half maximal inhibitory concentration (IC₅₀) for DOX is about 0.25 μg/mL. Bar 850 represents the drug carriers loaded with 0.25 μg/mL DOX, but not underwent the NIR irradiation after incubating with the cells. Therefore, the DOX was still encapsulated by the drug carriers, and the cell death rate was not high. Bar 860 represents the drug carriers loaded with 0.25 μg/mL DOX, and underwent the NIR irradiation after incubating with the cells. Comparing to the pure DOX of the same concentration, i.e. bar 840, which only caused 50% cell death, the drug carriers with the NIR irradiation had significant effects. With the result shown in FIG. 12C, not only the cells at the irradiation area, but also the cells in the area out of the irradiation were killed. The conditions for bars 870, 880, and 890 were to reduce the DOX concentration from 0.25 μg/mL to 0.05 μg/mL, and therefore the cell viabilities for the control of pure DOX, i.e. bar 870, and the drug carriers without the NIR irradiation, i.e. bar 880, were increased. However, it is noteworthy that although the dosage loaded by the drug carriers that encapsulated with 0.05 μg/mL DOX and underwent the NIR irradiation, i.e. bar 890, were only 0.2 times of the IC₅₀ for DOX, over 95% of the cancer cells were killed with the cooperation of the photothermal therapy.

The following conclusions may be obtained by the foregoing results, including (1): if the carrier is not loaded with drugs, only cells at the NIR irradiation area are killed; (2): the drug carrier of the present invention cooperates with the photothermal therapy, and about 95% of the cancer cells can be killed; and (3): the drug carrier with the cooperation of the photothermal therapy only requires 0.2 times of drug dosage, and cancer cells at the whole area can be killed. The cell viability is reduced to less than 10%. The excellent effects of combining the photothermal therapy with the chemotherapy can be seen from these results. Such progressed efficiency is because the drug carrier of the present invention has the following three features: (1) rapid cell targeting and phagocytosis by cells, (2) high thermal sensitivity and rapid drug release, and (3) the effect of the NIR irradiation on board area. Therefore, the drug carrier of the present invention combines high efficiency of phagocytosis and the photothermal therapy to accurately deliver drugs to targeted cells, and the drugs are released by the control of NIR irradiation. Such treatment cannot only achieve the same therapeutic effect with less drug dosage, but also enhance the ability of killing cancer cells.

### Experimental Example 7

In Experimental example 7, the targeting efficiency of the drug carrier of the present invention was tested by experiments on in vivo tumors (RG2 cells) of mice. The tumor temperature was also observed while undergoing NIR irradiation. Further, the effects on the volume of the tumors and the mass of the mice were observed through different conditions of treatment.

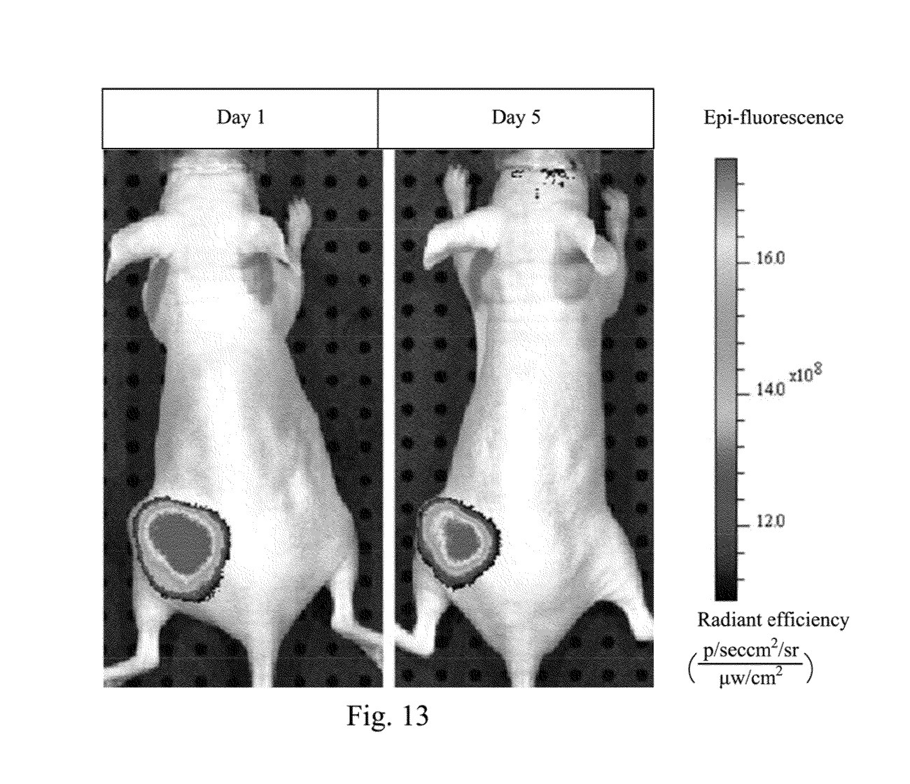

Referring to FIG. 13, which is fluorescence images of a mouse injected with drug carriers having fluorescence labels after 1 day and 5 days. 100 μL of 1 wt % drug carriers was intravenously injected into the mouse, which the drug carriers were labeled by Cy 5.5, and fluorescent signals were observed by an epi-fluorescence microscope. The fluorescent signals (Ex: 640 nm, Em: 710 nm) were observed on both day 1 and day 5, and the signals did not decreased, suggesting the success of targeting and the dosage accumulation of the drug carriers. The targeting efficiency of the drug carrier of the present invention is mainly controlled by the lactoferrin of the drug carrier, which can make the drug carrier quickly close to RG2 cells, and increase the phagocytic efficiency of the cancer cells.

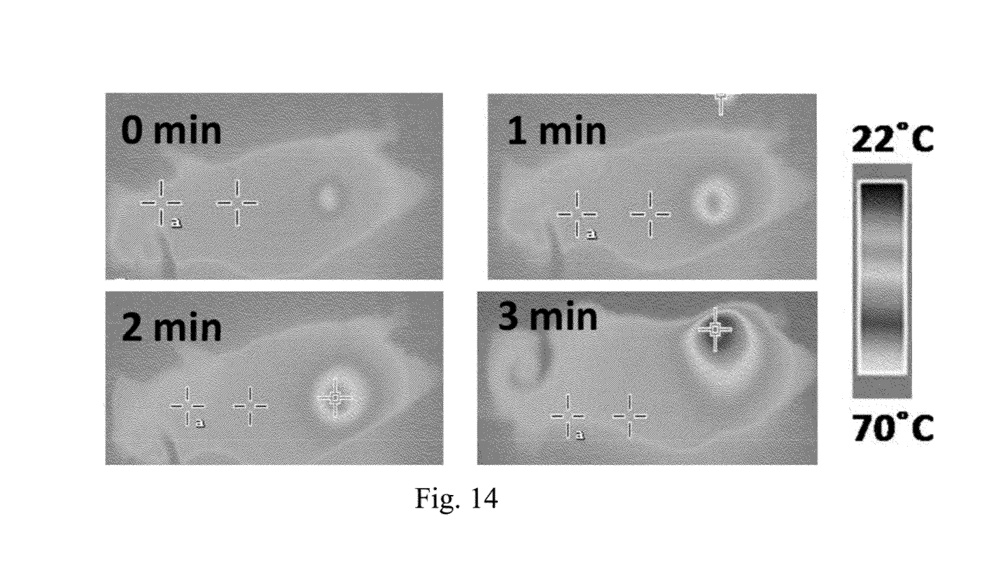

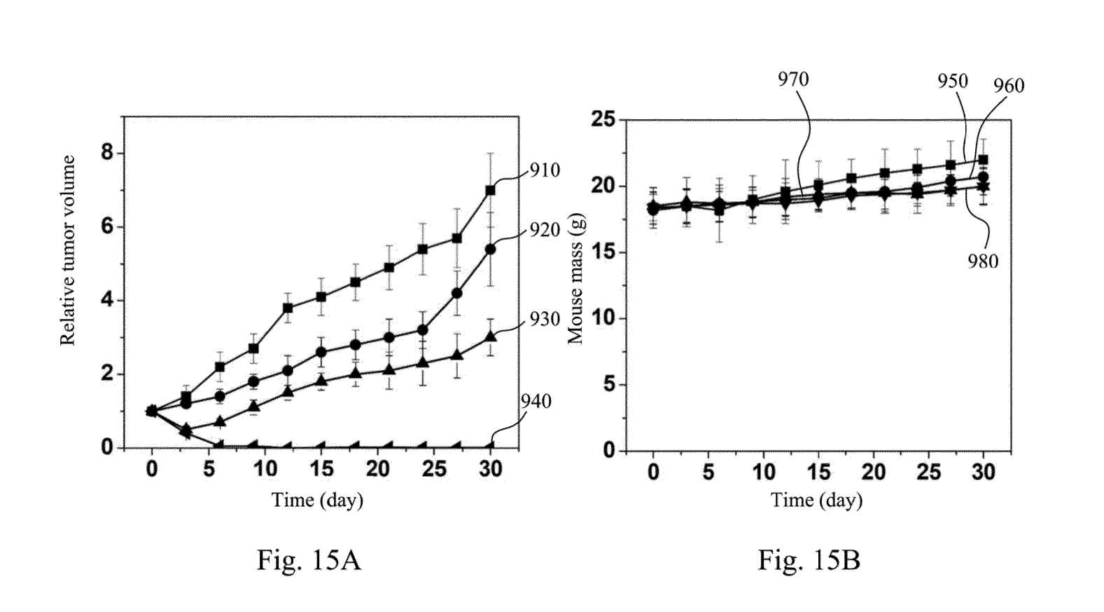

Referring to FIG. 14, which is infrared thermal images of a mouse injected with drug carriers according to an embodiment of the present invention, and irradiated by NIR for 0 minute, 1 minute, 2 minutes, and 3 minutes. The intensity of NIR irradiated at a tumor region of the mouse was 2 W/cm². As shown in FIG. 14, because the rGO of the drug carriers would absorb NIR, the temperature at the tumor region of the mouse increased as the irradiation duration increased. The tumor temperature was increased from about 31° C. to about 55° C. after being irradiated for 3 minutes. The increase in the tumor temperature of the mouse suggests that the drug carrier of the present invention does can be delivered to the tumor region in vivo.

Referring to FIG. 15A, which is a relative tumor volume vs. time graph of mice after treated by different conditions. The treating conditions were PBS+NIR irradiation, pure DOX+NIR irradiation, non-drug loaded carriers+NIR irradiation, and carriers loaded with DOX+NIR irradiation, and changes in the relative tumor volume along time of which are represented by lines 910, 920, 930, and 940, respectively. The relative tumor volume was observed after 24 hours later of the treatment, and used the tumor volume right after the treatment as a base. As shown in FIG. 15A, the tumor volumes for the treatments of both of the non-drug loaded carriers, i.e. line 930, and the carriers loaded with DOX, i.e. line 940, and underwent the NIR irradiation were significantly decreased. As for the treatments of the PBS+NIR irradiation, i.e. line 910, and the pure DOX+NIR irradiation, i.e. line 920, the tumor volumes were increased. These results confirm the effectiveness of in vivo therapy. However, after 3 days, the tumor of the mouse being treated by the non-drug loaded carriers+NIR irradiation, i.e. line 930, began to grow gradually. In comparison, the tumor of the mouse being treated by the carriers loaded with DOX, i.e. line 940, was nearly disappeared in the body, and there was no recurrence in next two months. The treatment of the PBS+NIR irradiation, i.e. line 910, could not treat the tumor because there was no drug being injected. The treatment of the pure DOX+NIR irradiation, i.e. line 920, was difficult to reach the tumor region to have the effect of therapy because the drug was not encapsulated in carriers. The treatment of the non-drug loaded carriers+NIR irradiation, i.e. line 930, could reduce the tumor volume at the beginning because the carriers could reach the tumor region, and incorporated with the photothermal therapy of NIR. However, the carries were not loaded with drug, so the tumor cells could not be killed completely, and the tumor began to grow after a period of time. Given the above, only combining the drug carrier of the present invention and the NIR irradiation can kill the tumor cells completely, and prevent the recurrence thereof.

Further, referring to FIG. 15B, which is a mass vs. time graph of mice after treated by different conditions. The treating conditions were PBS+NIR irradiation, pure DOX+NIR irradiation, non-drug loaded carriers+NIR irradiation, and carriers loaded with DOX+NIR irradiation, and changes in the mass of the mice along time of which are represented by lines 950, 960, 970, and 980, respectively. As shown in FIG. 15B, there was no obvious difference in mass, which suggests that the carrier having the lactoferrin and graphenes can significantly reduce the damage of anti-cancer drug to the body.

### Experimental Example 8

Under the stimulation of the NIR irradiation, the carriers can reach a temperature enough to kill the cancer cells, which is about 55° C. Therefore, it cannot be confirmed that whether combining the photothermal therapy and the chemotherapy is advantageous. Accordingly, Experimental example 8 is to discuss the effect of whether carriers loaded with drugs together with the NIR irradiation on treating tumor cells.

Three groups of nude mice with subcutaneous tumors were treated with three types of treatments respectively, including injection of PBS together with the NIR irradiation as a control, injection of non-drug loaded carriers together with the NIR irradiation (carriers+NIR irradiation), and injection of drug carriers loaded with DOX together with the NIR irradiation (drug carriers+NIR irradiation), wherein the intensity of the NIR irradiated was 2 W/cm². After treating for three days, the tumors of the groups of carriers+NIR irradiation and drug carriers+NIR irradiation developed into black scab, which there was no obvious change in morphology of the control. However, after seven days, the tumor of the group of carriers+NIR irradiation began to grow again, which the tumor of the group of drug carriers+NIR irradiation continuously decreased, and disappeared after one month. In the final observation, the tumor of the group of carriers+NIR irradiation recurred at the area around the NIR irradiation area. Although there was no recurrence at the center of the tumor cells, which has developed into scab, the tumor cells migrated to the area surrounding the NIR irradiation area to form new tumors and continue to grow.

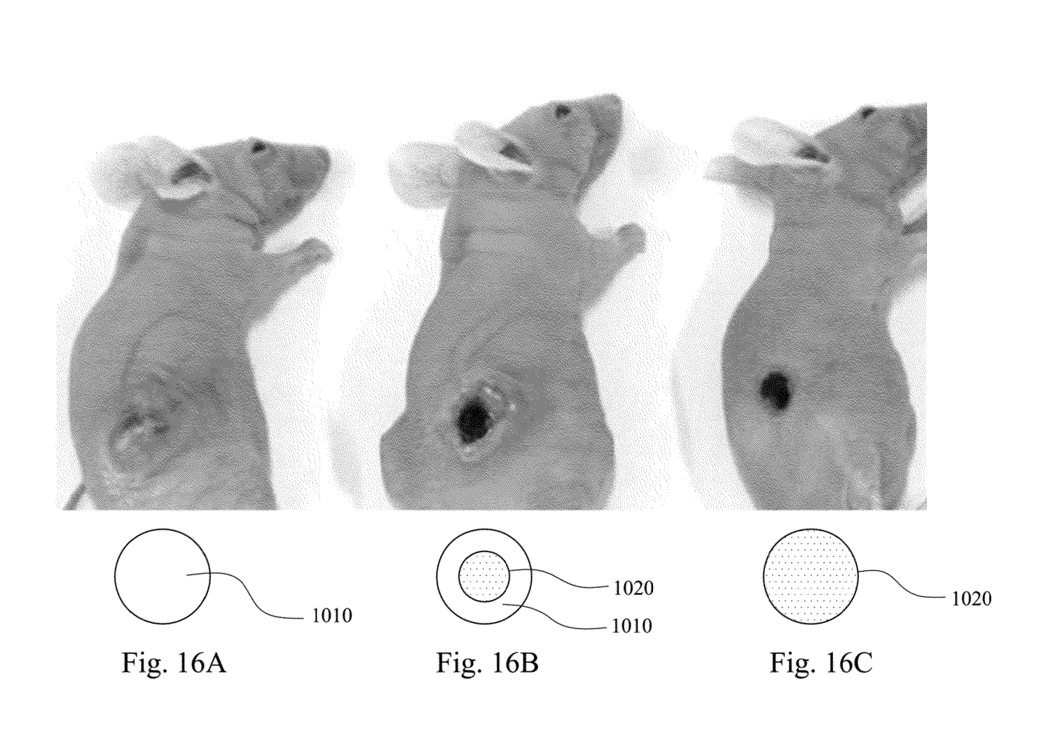

Referring to FIGS. 16A through 16C, which are pictures of mice and schematic graphs of growth status for tumor cells treated by different conditions after 14 days, wherein, in the schematic graphs of growth status for tumor cells, white parts represent living tumor cells 1010, and dotted parts represent dead tumor cells 1020. FIG. 16A shows the control of being treated by PBS and the NIR irradiation, which suggests that the tumor cells would not be killed for the mouse only irradiated by NIR without injected carriers or drug carries. FIG. 16B shows the group of carriers+NIR irradiation, which was injected with non-drug loaded carriers and irradiated by NIR. As shown in FIG. 16B, although the tumor cells at the irradiation area were killed with the injection of the carriers and the NIR irradiation, the tumor cells would migrate to the area surrounding the irradiation area and form new tumors after a period of time due to the absence of drug in the carriers. FIG. 16C shows the group of drug carriers+NIR irradiation, which was injected with drug carriers loaded with DOX and irradiated by NIR. As shown in FIG. 16C, the combination of the photothermal therapy and the chemotherapy could not only kill the tumor cells at the irradiation area, but also kill the tumor cells at the area surrounding the irradiation area due to the diffusion of DOX released by the NIR irradiation, which could prevent the recurrence of the tumor.

The above result shows that it is hard to completely eliminate the tumor cells for the treatment of the group of carriers+NIR irradiation, which the tumor is only treated by the photothermal therapy, and the tumor cells are prone to migrate to the area that is not irradiated by NIR to grow continuously. However, when combing the photothermal therapy and the chemotherapy, which is the group of drug carriers+NIR irradiation, there is no recurrence of tumor even after one month since the treatment. This suggests that the anti-cancer drug can eliminate the tumor cells at not only the irradiation area, but also the area near the irradiation area. The reason for this result is that the drug carrier of the present invention has good thermal sensitive property, and can increase the tumor temperature to about 50° C., which can kill the cancer cells, and promote the absorption and release of drugs.

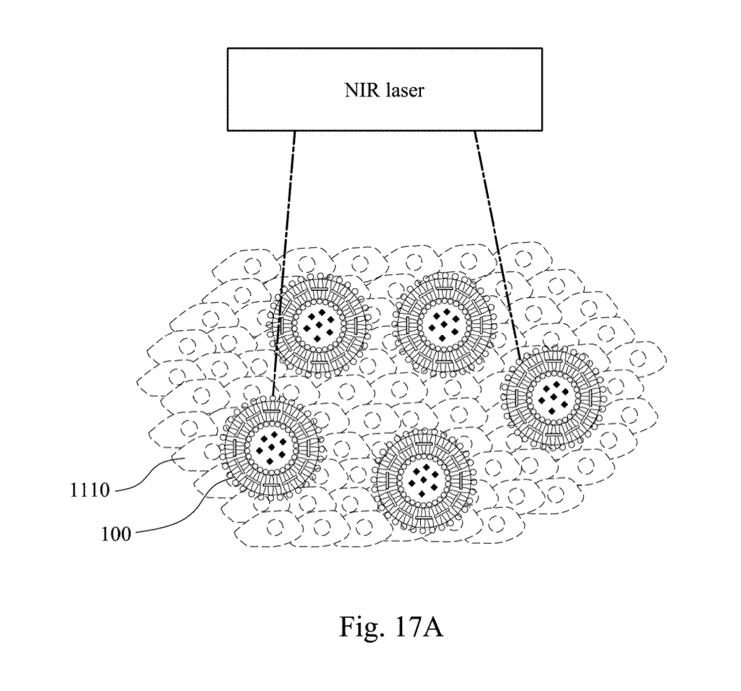

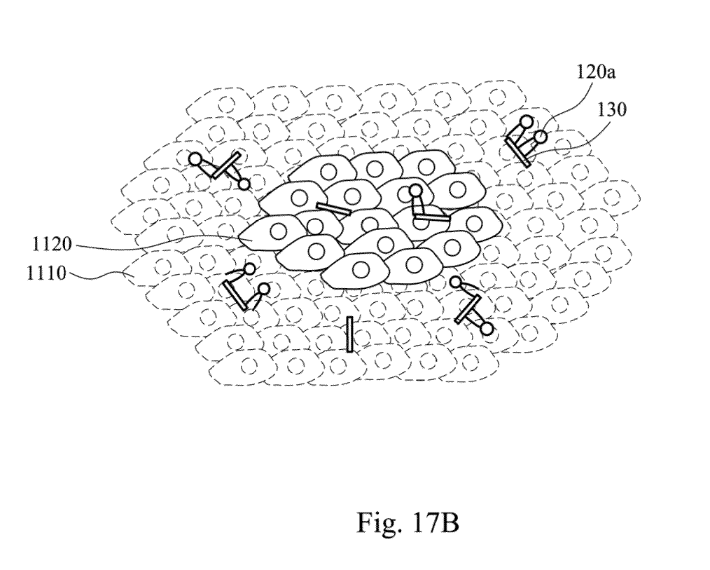

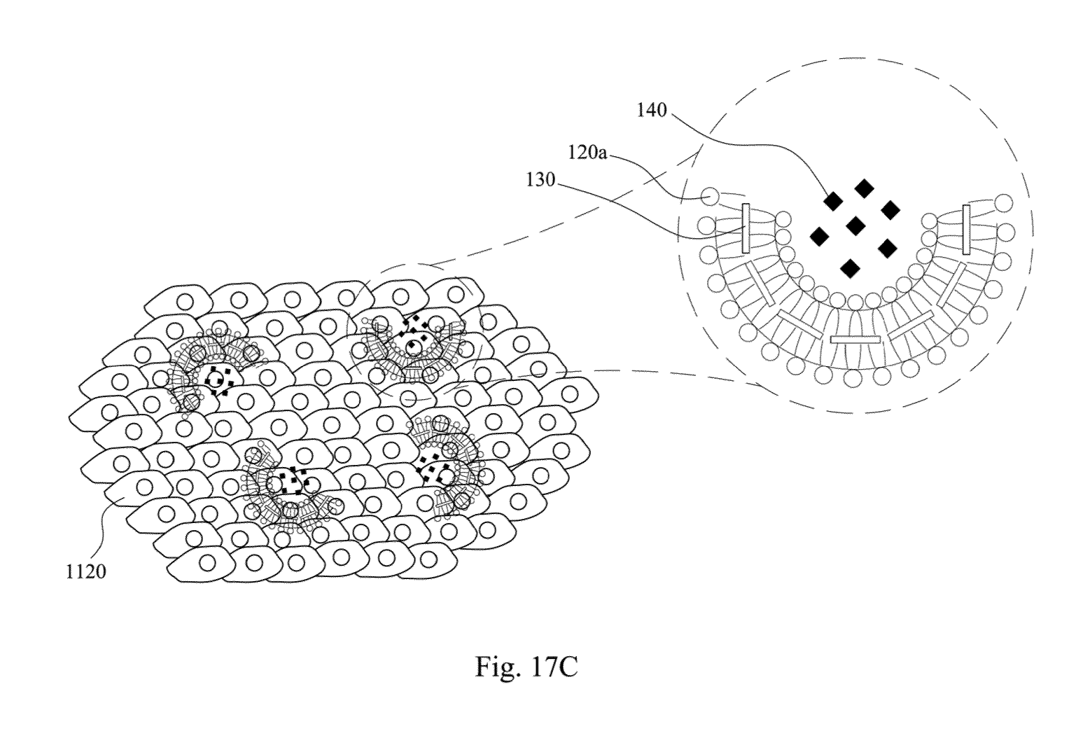

The schematic graphs of carriers under the NIR irradiation treatment are shown in FIGS. 17A through 17C, which the dotted lines represent living tumor cells 1110, and the solid lines represent dead tumor cells 1120. FIG. 17A is a schematic graph of carriers at tumor cells, i.e. the living tumor cells 1110, irradiated by NIR laser, and the carriers are represented by the drug carrier 100 in this figure. When the carries are the non-drug loaded carriers, the growth status for the tumor cells after irradiated by NIR is shown in FIG. 17B, wherein the carriers has a structure similar to the drug carrier 100, only the aqueous solution does not include drugs. Because the carriers include the graphenes 130, the graphenes 130 would absorb NIR and cause the deformation or collapse of the carriers, and the carriers are thus broke into fragments, which some lactoferrin 120a may still attaches on the graphenes 130. Also, the temperature of the carriers is increased enough to kill the tumor cells, such as 55° C., due to the absorption of NIR. Therefore, the tumor cells at the irradiation area would be killed, i.e. dead tumor cells 1120, while the tumor cells outside of the irradiation area would still alive, i.e. living tumor cells 1110. This kind treatment only has an effect of the photothermal therapy, and the tumor cells cannot be killed completely. When the carriers are loaded with a drug, such as the drug carriers 100, the growth status for the tumor cells after irradiated by NIR is shown in FIG. 17C. Because the drug carriers 100 include the graphenes 130, the graphenes 130 would absorb NIR and cause the deformation or collapse of the drug carriers 100, and the drug carriers 100 are thus broke into fragments to release the drug 140. The released drug 140 would diffuse to the area that is not irradiated by NIR, and therefore the tumor cells at both of the irradiation area and the area near the irradiation area would be killed, i.e. dead tumor cell 1120. As described above, the drug carrier of the present invention combines the photothermal therapy and the chemotherapy, which can not only kill the tumor cells, but also prevent the recurrence of the tumor.

### Preparation of Drug Carriers

The method for preparing drug carriers according to another embodiment of the present invention includes the following steps:
1. 2 mg of lactoferrin (Lf) was dissolved in 100 μL of de-ionized water as a first aqueous solution.
2. 4 mg of iron oxide was dissolved in 250 μL of chloroform.
3. 0.75 mg of hydrophobic rGO was dispersed in the solution of step 2 as an oil solution.
4. The first aqueous solution of step 1 was mixed with the oil solution of step 3, and emulsified by ultrasonifucation (20 kHz, 130 W) for 30 seconds to form a water-in-oil (w/o) first emulsion.
5. 8 mg of lactoferrin was dissolved in 400 μL of de-ionized water as a second aqueous solution.
6. The second aqueous solution of step 5 was emulsified with the first emulsion of step 4 using similar process described in step 4 to form a water-in-oil-in-water (w/o/w) second emulsion.
7. The second emulsion of step 6 was concentrated and volatilized under vacuum to remove the chloroform and to form non-toxic drug carriers.
8. The products of step 7 were dialyzed by a dialysis membrane with a pore size of 140K molecular weight in de-ionized water to remove unreacted lactoferrin, iron oxide, and impurities to obtain the drug carriers with the graphene, iron oxide, and lactoferrin (Graphene/iron oxide@lactoferrin drug carrier).

The iron oxide was oil-soluble Fe₃O₄ nanoparticles, which is coated with oleic acid, and the preparing method can refer to Sun, S. H., et al. Journal of the American Chemical Society, 2004, 126(1), 273-279. A drug was dissolved in the first aqueous solution in step 1 to form the drug carriers 400 shown in FIG. 4, or dissolved in the oil solution in step 3 to form the drug carriers 500 shown in FIG. 5.

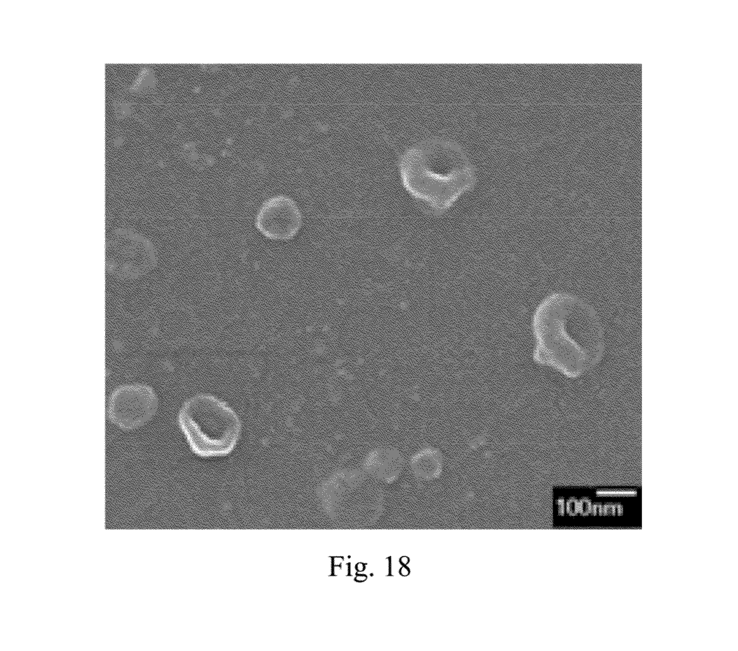

Referring to FIG. 18, which is a scanning electron microscope (SEM) image of drug carriers prepared by the aforementioned methods. The lactoferrin concentration for the drug carries was 0.05 wt %, and the iron oxide concentration was 16 mg/mL while preparing the drug carriers. The carrier has to be prepared under a specific ratio of the lactoferrin and the iron oxide to have complete morphology. The concentration of the lactoferrin can affect the size of the drug carrier, and the higher concentration would form a carrier with a thicker shell. The size of the drug carrier can affect the encapsulation efficiency (EE %) of the drug. The larger drug carrier has larger internal hollow volume, and thus has better encapsulation efficiency. In addition, when the iron oxide is less than 6 mg/mL, no carriers could be formed. The concentration of the iron oxide has to be controlled in a range of 5-30 mg/mL to form carriers with complete spheres. In order to form carriers with complete structures, the concentration of the graphene may be reduced, and controlled in a range of 0.01-0.2 wt %.

The following experiments in Experimental examples 9 and 10 were conducted by drug carriers having a structure of the drug carrier 500.

### Experimental Example 9

Magnetic guide: In order to test the efficiency of the magnetic guide in tumor targeting, a magnet was used as the magnetic guide at the tumor region of a mouse for 1 hour, 4 hours, and 8 hours to observe the effect of the magnetic guide on drug carrier accumulation after three different durations attracted by the magnet.

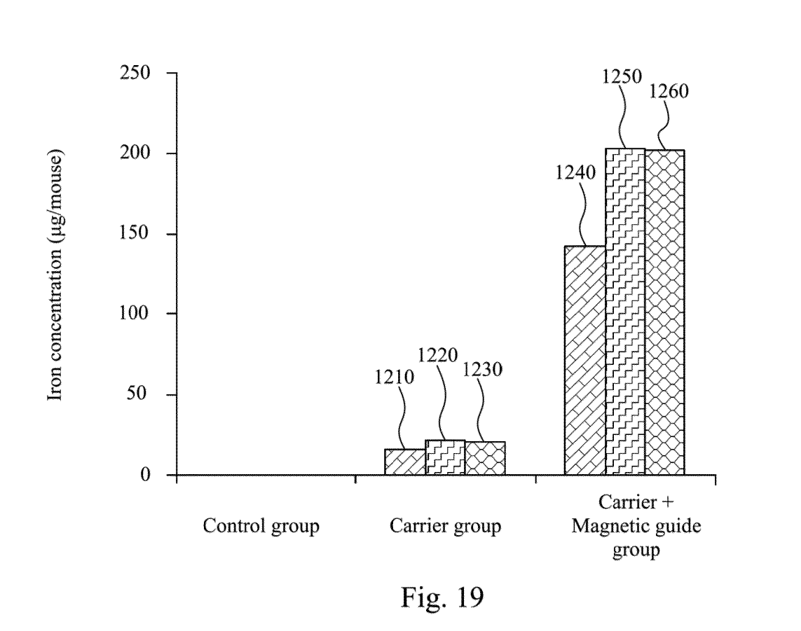

Referring to FIG. 19, which is a bar chart for iron concentration in tumor regions of a control group, which was without carriers, a carrier group, which was injected with drug carriers and without magnetic guide, and a carrier+magnetic guide group, which was injected with drug carriers and with magnetic guide, after different times. Bars 1210, 1220, and 1230 represent the iron concentration in the tumor region of the carrier group after 1, 4, and 8 hour(s), respectively. Bars 1240, 1250, and 1260 represent the iron concentration in the tumor region of the camier+magnetic guide group after 1, 4, and 8 hour(s), respectively. There was no iron oxide in the tumor region for the control group because the control group was not injected with the drug carriers. As shown in FIG. 19, the iron concentrations in the tumor regions of the carrier group and the carrier+magnetic guide group were increase over time, which suggests that the source of the iron oxide came from the drug carriers comparing to the control group, which was not injected with the drug carriers. The drug carriers not guided by the magnet can still reach the tumor region, but the amount of the drug carriers reaching the tumor region can be largely improved with the magnetic guide.

### Experimental Example 10

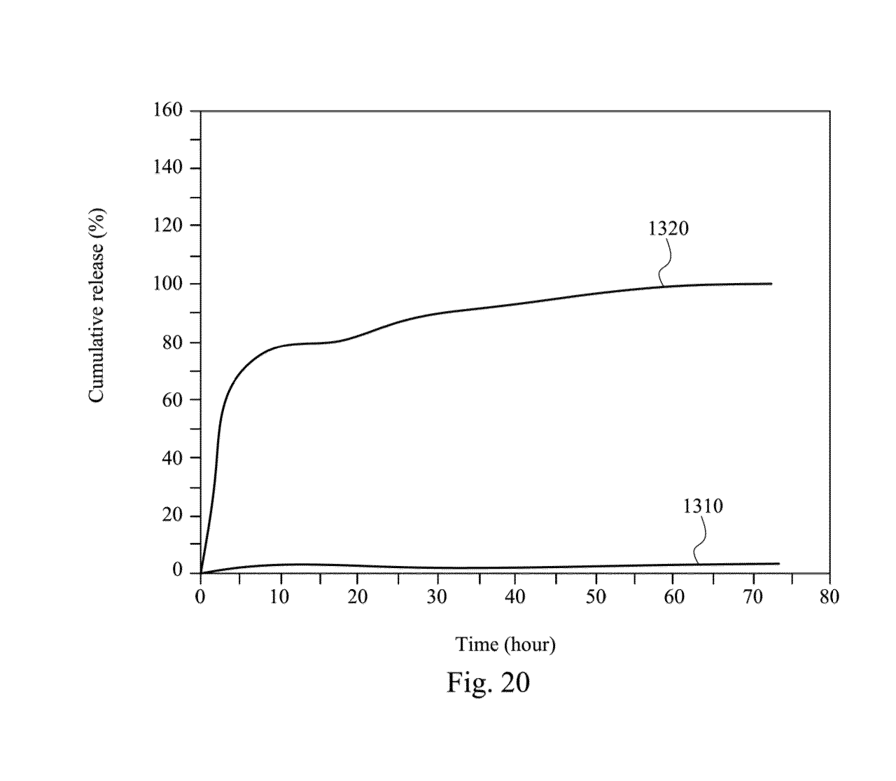

Experimental example 10 was to test the drug release ability of the drug carriers loaded with curcumin (Cur), which were prepared by the aforementioned method. Referring to FIG. 20, which is a cumulative release vs. time graph of the drug carriers under different environments, wherein lines 1310 and 1320 represent the cumulative release of the drug over time for the drug carriers in 40% dimethyl sulfoxide (DMSO)+60% water and in 100% water respectively. As shown in FIG. 20, the drug release for the drug carriers loaded with Cur had two stages in 40% DMSO+60% water. The release rate was faster at the first stage, and became slower at the latter stage. The drug was barely released when the drug carriers were in 100% water.

### Preparation of Drug Carriers

According to an embodiment of the present invention, the protein shell of the drug carrier is made of albumin, which has high biocompatibility. In this embodiment, the albumin was used to replace the lactoferrin used in the abovementioned embodiments, and a hydrophilic drug, DOX, was used as the bioactive agent. The method for preparing the drug carriers having the albumin includes the following steps:
1. 0.75 mg of hydrophobic rGO was dispersed in 250 μL of chloroform as an oil solution.
2. 2 mg of bovine serum albumin (BSA) and 0.5 mg of doxorubicin (DOX) were dissolved in 100 μL of de-ionized water as a first aqueous solution.
3. The first aqueous solution of step 2 was mixed with the oil solution of step 1, and emulsified by ultrasonifucation (20 kHz, 130 W) for 1 minute to form a water-in-oil (w/o) first emulsion.
4. 400 μL of 2% BSA was prepared as a second aqueous solution.
5. The second aqueous solution of step 4 was emulsified with the first emulsion of step 3 using similar process described in step 3 to form a water-in-oil-in-water (w/o/w) second emulsion.
6. The second emulsion of step 5 was stirred at room temperature until the organic solvent was completely evaporated.
7. The products of step 6 were centrifuged to remove unreacted materials, and were dispersed in double-distilled water to obtain the drug carriers. The structure of the drug carriers may refer to the drug carrier 100 shown in FIG. 1.

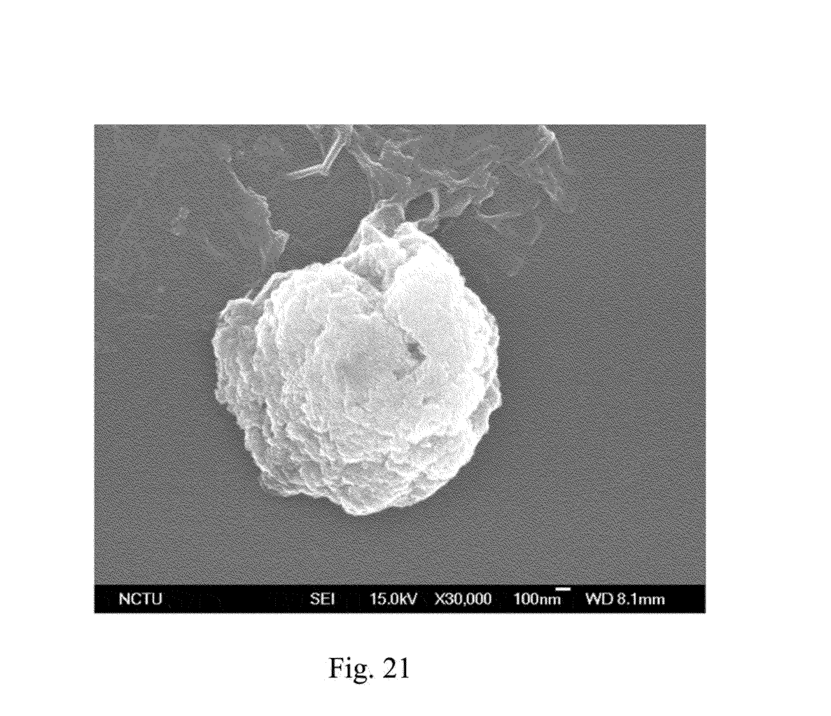

The obtained drug carriers were observed by an electron microscope. Referring to FIG. 21, which shows a scanning electron microscope (SEM) image of the drug carriers. As shown in FIG. 21, the form, structure and size of the drug carriers can be observed. The components of the drug carrier have to be controlled in a specific ratio to form the drug carrier with a more complete structure. The diameter of the rGO has to be controlled in a range of 20-200 nm, and the concentration of the rGO has to be controlled in a range of 0.05-2 wt % to form carriers with complete spheres. In addition, the concentration of the albumin can affect the size of the drug carrier, and the higher concentration would form a carrier with a thicker shell. The size of the drug carrier can affect the encapsulation efficiency (EE %) of the drug. The larger drug carrier has larger internal hollow volume, and thus has better encapsulation efficiency. The drug carriers have a diameter in a range of 600-2000 nm.

### Preparation of Drug Carriers

According to an embodiment of the present invention, the protein shell of the drug carrier is made of silk protein, which has high biocompatibility. In this embodiment, the silk protein was used to replace the lactoferrin used in the abovementioned embodiments, and a hydrophilic nerve growth factor was used as the bioactive agent. The method for preparing the drug carriers having the silk protein includes the following steps:
1. 0.75 mg of hydrophobic rGO was dispersed in 250 μL of chloroform as an oil solution.
2. 0.05 mg of silk protein and 0.5 mg of nerve growth factor (NGF) were dissolved in 100 μL of de-ionized water as a first aqueous solution.
3. The first aqueous solution of step 2 was mixed with the oil solution of step 1, and emulsified by ultrasonifucation (20 kHz, 130 W) for 1 minute to form a water-in-oil (w/o) first emulsion.
4. 400 μL of 0.05% silk protein was prepared as a second aqueous solution.
5. The second aqueous solution of step 4 was emulsified with the first emulsion of step 3 using similar process described in step 3 to form a water-in-oil-in-water (w/o/w) second emulsion.
6. The second emulsion of step 5 was stirred at room temperature until the organic solvent was completely evaporated.
7. The products of step 6 were centrifuged to remove unreacted materials, and were dispersed in double-distilled water to obtain the drug carriers. The structure of the drug carriers may refer to the drug carrier 100 shown in FIG. 1.

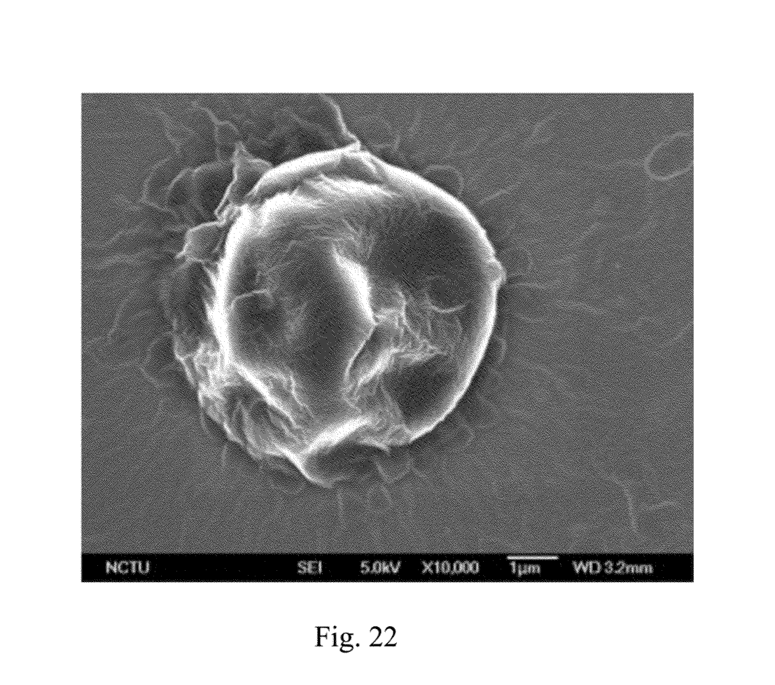

The obtained drug carriers were observed by an electron microscope. Referring to FIG. 22, which shows a scanning electron microscope (SEM) image of the drug carriers. As shown in FIG. 22, the form, structure and size of the drug carriers can be observed. The components of the drug carrier have to be controlled in a specific ratio to form the drug carrier with a more complete structure. The diameter of the rGO has to be controlled in a range of 50-400 nm, and the concentration of the rGO has to be controlled in a range of 0.5-4 wt % to form carriers with complete spheres. In addition, the concentration of the silk protein can affect the size of the drug carrier, and the higher concentration would form a carrier with a thicker shell. The size of the drug carrier can affect the encapsulation efficiency (EE %) of the drug. The larger drug carrier has larger internal hollow volume, and thus has better encapsulation efficiency. The drug carriers have a diameter in a range of 800-3000 nm.

### Experimental Example 11

Experimental example 11 was to discuss the effects of electrical stimulation on the drug carrier of the present invention.

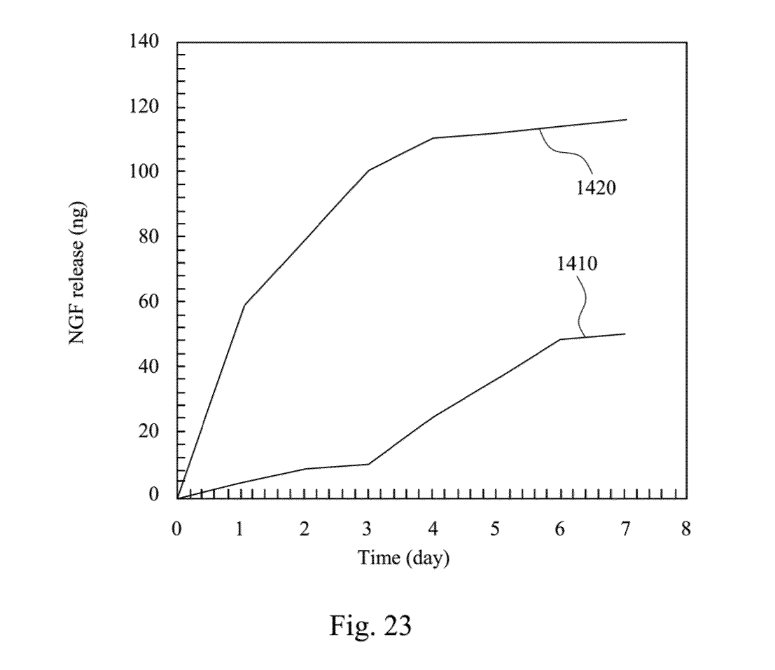

In Experimental example 11, an electrical field was applied to the drug carriers with silk protein and nerve growth factor (NGF), and the NGF release of the drug carriers was observed. Referring to FIG. 23, which illustrates a nerve growth factor release vs. time graph of an embodiment and a comparative example, which line 1410 represents drug carriers without electrical stimulation as the comparative example, and line 1420 represents drug carriers underwent electrical stimulation as the embodiment. As shown in FIG. 23, the NGF release of the drug carriers underwent the electrical stimulation was much higher than that of the drug carriers without electrical stimulation. These results are because the graphene is susceptible to the electrical stimulation. When applying external electrical field to the drug carrier, the graphene is stimulated to vibrate, which leads to the structural destruction of the drug carrier, and the NGF is then released. Experimental example 11 proves that the protein-graphene nanocomposite drug carrier of the present invention can also encapsulate a growth factor that promotes cell proliferation and differentiation to further applied to tissue engineering and regeneration.

The drug carrier of the present invention has longer circulation in the body, and can reach the designated therapeutic areas because the protein can be recognized by the immune system in the body. Further, the graphene can stabilize the structure of the carrier, and by using its characteristic of absorbing NIR or being susceptible to electrical stimulation, the timing and amount of the release of the bioactive agent of the drug carrier of the present invention can be controlled by NIR or electrical stimulation. The drug carrier of the present invention may further include iron oxide, which equips the drug carrier with a function of physical magnetic guide in addition to the effect of photothermal therapy. The drug carrier is guided by the magnetic guide to reach the designated therapeutic areas, which the targeting of the drug carrier toward those areas can be enhanced. The drug carrier of the present invention has an easy preparing process, and is a multifunctional, non-toxic, hollow, protein nanocomposite drug carrier. Different pharmaceutical purposes can be achieved by altering the material encapsulated by the drug carrier. For instance, efficacy of chemotherapy can be achieved by encapsulating anti-cancer drugs, efficacy of tissue repairing can be achieved by encapsulating DNA or other repairing proteins, and a purpose of bioimaging can be achieved by encapsulating fluorescent materials. The drug carrier of the present invention has great potential for clinical medicine.

It will be apparent to those ordinarily skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A drug carrier, comprising:
an aqueous solution;
a protein shell enclosing the aqueous solution and comprising at least one hydrophilic/hydrophobic layer;
a plurality of graphenes dispersed in the protein shell; and
a bioactive agent in the aqueous solution and/or the protein shell.

2. The drug carrier of claim 1, wherein the protein shell is made of a protein, which is amphiphilic lactoferrin, albumin or silk protein.

3. The drug carrier of claim 2, wherein the protein has a concentration of about 1-5 wt % in the drug carrier.

4. The drug carrier of claim 1, wherein the graphenes have a concentration of about 0.01-4 wt % in the drug carrier.

5. The drug carrier of claim 1, wherein the graphenes are reduced graphene oxides.

6. The drug carrier of claim 1, wherein the graphenes have a diameter of about 20-400 nm.

7. The drug carrier of claim 1, further comprising iron oxide dispersed in the protein shell.

8. The drug carrier of claim 1, wherein the drug carrier has a diameter of about 100-4000 nm.

9. The drug carrier of claim 1, wherein the bioactive agent in the aqueous solution is a hydrophilic agent.

10. The drug carrier of claim 9, wherein the hydrophilic agent is antitumor drug, protein drug, antibiotic or growth factor.

11. The drug carrier of claim 10, wherein the antitumor drug is doxorubicin (DOX) or cisplatin (CDDP).

12. The drug carrier of claim 1, wherein the bioactive agent in the protein shell is a hydrophobic agent.

13. The drug carrier of claim 12, wherein the hydrophobic agent is curcumin (Cur) or paclitaxel.
